SEQUENCE LISTING (107 sequences)

SEQ ID NO: 1 - DNA, 26 bp, artificial
  oligonucleotide for amplifying putative gene located on Chromosome 5H
gcccgcgtcatcactagtaaatcttg

SEQ ID NO: 2 - DNA, 26 bp, artificial
  oligonucleotide for amplifying putative gene located on Chromosome 5H
gcccgcgtcatcactagtaaatctta

SEQ ID NO: 3 - DNA, 23 bp, artificial
  oligonucleotide for amplifying putative gene located on Chromosome 5H
ggcggcggagaaaaagtaatggt

SEQ ID NO: 4 - DNA, 28 bp, artificial
  oligonucleotide for amplifying putative gene located on Chromosome 5H
cccgcgaaatcatcactagtaaatcttg

SEQ ID NO: 5 - DNA, 28 bp, artificial
  oligonucleotide for amplifying putative gene located on Chromosome 5H
cccgcgaaatcatcactagtaaatctta

SEQ ID NO: 6 - DNA, 23 bp, artificial
  oligonucleotide for amplifying putative gene located on Chromosome 5H
gcggcgggagaaaaagtaatggt

SEQ ID NO: 7 - DNA, 30 bp, artificial
  oligonucleotide for amplifying putative gene located on Chromosome 5H
ggcgcagtaaatcatcactagtaaatcttg

SEQ ID NO: 8 - DNA, 30 bp, artificial
  oligonucleotide for amplifying putative gene located on Chromosome 5H
ggcgcagtaaatcatcactagtaaatctta

SEQ ID NO: 9 - DNA, 25 bp, artificial
  oligonucleotide for amplifying fragment of nicotinate phosphorosyltransferase-like gene
cctgccttgttctggacgttttcat

SEQ ID NO: 10 - DNA, 21 bp, artificial
  oligonucleotide for amplifying fragment of nicotinate phosphorosyltransferase-like gene
gcgccggccgaatcagtttac

SEQ ID NO: 11 - DNA, 21 bp, artificial
  oligonucleotide for amplifying fragment of nicotinate phosphorosyltransferase-like gene
gcgccggccgaatcagtttag

SEQ ID NO: 12 - DNA, 21 bp, artificial
  oligonucleotide for amplifying fragment of nicotinate phosphorosyltransferase-like gene
ggcggctgaattcacaggctg

SEQ ID NO: 13 - DNA, 21 bp, artificial
  oligonucleotide for amplifying fragment of nicotinate phosphorosyltransferase-like gene
gcccgcgccgaatcagtttac

SEQ ID NO: 14 - DNA, 21 bp, artificial
  oligonucleotide for amplifying fragment of nicotinate phosphorosyltransferase-like gene
gcccgcgccgaatcagtttag

SEQ ID NO: 15 - DNA, 22 bp, artificial
  oligonucleotide for amplifying fragment of nicotinate phosphorosyltransferase-like gene
gcggcactgaattcacaggctg

SEQ ID NO: 16 - DNA, 21 bp, artificial
  oligonucleotide for amplifying fragment of nicotinate phosphorosyltransferase-like gene
ccgcccgccgaatcagtttac

SEQ ID NO: 17 - DNA, 21 bp, artificial
  oligonucleotide for amplifying fragment of nicotinate phosphorosyltransferase-like gene
ccgcccgccgaatcagtttag

SEQ ID NO: 18 - DNA, 23 bp, artificial
  oligonucleotide for amplifying fragment of nicotinate phosphorosyltransferase-like gene
ccgcaactgaattcacaggctga

SEQ ID NO: 19 - DNA, 26 bp, artificial
  oligonucleotide for amplifying putative gene located on Chromosome 5H
tgtgtctgaacttgcatttgatgacg

SEQ ID NO: 20 - DNA, 27 bp, artificial
  oligonucleotide for amplifying putative gene located on Chromosome 5H
cctctctttgtgctctcaacttgtcca

SEQ ID NO: 21 - DNA, 26 bp, artificial
  oligonucleotide for amplifying putative gene located on Chromosome 5H
gcccgcgtcatcactagtaaatcttg

SEQ ID NO: 22 - DNA, 26 bp, artificial
  oligonucleotide for amplifying putative gene located on Chromosome 5H
gcccgcgtcatcactagtaaatctta

SEQ ID NO: 23 - DNA, 23 bp, artificial
  oligonucleotide for amplifying putative gene located on Chromosome 5H
ggcggcggagaaaaagtaatggt

SEQ ID NO: 24 - DNA, 26 bp, artificial
  oligonucleotide for amplifying putative gene located on Chromosome 5H
tgtgtctgaacttgcatttgatgacg

SEQ ID NO: 25 - DNA, 27 bp, artificial
  oligonucleotide for amplifying putative gene located on Chromosome 5H
cctctctttgtgctctcaacttgtcca

SEQ ID NO: 26 - DNA, 26 bp, artificial
  oligonucleotide for amplifying putative gene located on Chromosome 5H
gcccgcgtcatcactagtaaatcttg

SEQ ID NO: 27 - DNA, 26 bp, artificial
  oligonucleotide for amplifying putative gene located on Chromosome 5H
gcccgcgtcatcactagtaaatctta

SEQ ID NO: 28 - DNA, 23 bp, artificial
  oligonucleotide for amplifying putative gene located on Chromosome 5H
ggcggcggagaaaaagtaatggt

SEQ ID NO: 29 - DNA, 20 bp, artificial
  oligonucleotide for amplifying fragment of nicotinate phosphorosyltransferase-like gene
ctactggaaggccggcaagc

SEQ ID NO: 30 - DNA, 24 bp, artificial
  oligonucleotide for amplifying fragment of nicotinate phosphorosyltransferase-like gene
cgcataaacctcaacatctgagca

SEQ ID NO: 31 - DNA, 21 bp, artificial
  oligonucleotide for amplifying fragment of nicotinate phosphorosyltransferase-like gene
gcgccggccgaatcagtttac

SEQ ID NO: 32 - DNA, 21 bp, artificial
  oligonucleotide for amplifying fragment of nicotinate phosphorosyltransferase-like gene
gcgccggccgaatcagtttag

SEQ ID NO: 33 - DNA, 21 bp, artificial
  oligonucleotide for amplifying fragment of nicotinate phosphorosyltransferase-like gene
ggcggctgaattcacaggctg

SEQ ID NO: 34 - DNA, 20 bp, artificial
  oligonucleotide for amplifying fragment of nicotinate phosphorosyltransferase-like gene
ctactggaaggccggcaagc

SEQ ID NO: 35 - DNA, 24 bp, artificial
  oligonucleotide for amplifying fragment of nicotinate phosphorosyltransferase-like gene
cgcataaacctcaacatctgagca

SEQ ID NO: 36 - DNA, 28 bp, artificial
  oligonucleotide for amplifying fragment of nicotinate phosphorosyltransferase-like gene
cccgtcgcgtgacaactaaaattataca

SEQ ID NO: 37 - DNA, 28 bp, artificial
  oligonucleotide for amplifying fragment of nicotinate phosphorosyltransferase-like gene
cccgtcgcgtgacaactaaaattatact

SEQ ID NO: 38 - DNA, 23 bp, artificial
  oligonucleotide for amplifying fragment of nicotinate phosphorosyltransferase-like gene
ggccgtcgctcatacaagtggaa

SEQ ID NO: 39 - DNA, 22 bp, artificial
  oligonucleotide for amplifying fragment of nicotinate phosphorosyltransferase-like gene
tacttcctcgagaagtacgccg

SEQ ID NO: 40 - DNA, 20 bp, artificial
  oligonucleotide for amplifying fragment of nicotinate phosphorosyltransferase-like gene
gtagagcgtgatcaccgtgg

SEQ ID NO: 41 - DNA, 23 bp, artificial
  oligonucleotide for amplifying fragment of nicotinate phosphorosyltransferase-like gene
gcgccaaagcttctgccagtctc

SEQ ID NO: 42 - DNA, 23 bp, artificial
  oligonucleotide for amplifying fragment of nicotinate phosphorosyltransferase-like gene
gcgccaaagcttctgccagtgag

SEQ ID NO: 43 - DNA, 21 bp, artificial
  oligonucleotide for amplifying fragment of nicotinate phosphorosyltransferase-like gene
gcgtgccagcgagaaggtgag

SEQ ID NO: 44 - DNA, 20 bp, artificial
  oligonucleotide for amplifying fragment of nicotinate phosphorosyltransferase-like gene
ctactggaaggccggcaagc

SEQ ID NO: 45 - DNA, 24 bp, artificial
  oligonucleotide for amplifying fragment of nicotinate phosphorosyltransferase-like gene
cgcataaacctcaacatctgagca

SEQ ID NO: 46 - DNA, 20 bp, artificial
  oligonucleotide for amplifying fragment of nicotinate phosphorosyltransferase-like gene
gcgccggccgaatcagtttg

SEQ ID NO: 47 - DNA, 21 bp, artificial
  oligonucleotide for amplifying fragment of nicotinate phosphorosyltransferase-like gene
ggcggctgaattcacaggctg

SEQ ID NO: 48 - DNA, 3 bp, artificial
  Tag region
gcg

SEQ ID NO: 49 - DNA, 3 bp, artificial
  Tag region
cgc

SEQ ID NO: 50 - DNA, 3 bp, artificial
  Tag region
ggc

SEQ ID NO: 51 - DNA, 3 bp, artificial
  Tag region
ccg

SEQ ID NO: 52 - DNA, 3 bp, artificial
  Tag region
gcc

SEQ ID NO: 53 - DNA, 4 bp, artificial
  Tag region
gcgg

SEQ ID NO: 54 - DNA, 4 bp, artificial
  Tag region
ggcg

SEQ ID NO: 55 - DNA, 4 bp, artificial
  Tag region
gcgc

SEQ ID NO: 56 - DNA, 4 bp, artificial
  Tag region
gggc

SEQ ID NO: 57 - DNA, 4 bp, artificial
  Tag region
gccg

SEQ ID NO: 58 - DNA, 4 bp, artificial
  Tag region
ggcc

SEQ ID NO: 59 - DNA, 4 bp, artificial
  Tag region
gcgg

SEQ ID NO: 60 - DNA, 4 bp, artificial
  Tag region
cgcg

SEQ ID NO: 61 - DNA, 4 bp, artificial
  Tag region
ccgc

SEQ ID NO: 62 - DNA, 4 bp, artificial
  Tag region
cggc

SEQ ID NO: 63 - DNA, 4 bp, artificial
  Tag region
cccg

SEQ ID NO: 64 - DNA, 4 bp, artificial
  Tag region
cgcc

SEQ ID NO: 65 - DNA, 4 bp, artificial
  Tag region
ccgg

SEQ ID NO: 66 - DNA, 7 bp, artificial
  Tag region
gcccgcg

SEQ ID NO: 67 - DNA, 8 bp, artificial
  Tag region
ggcggcgg

SEQ ID NO: 68 - DNA, 6 bp, artificial
  Tag region
cccgcg

SEQ ID NO: 69 - DNA, 5 bp, artificial
  Tag region
ggcgc

SEQ ID NO: 70 - DNA, 6 bp, artificial
  Tag region
gcgccg

SEQ ID NO: 71 - DNA, 5 bp, artificial
  Tag region
gcccg

SEQ ID NO: 72 - DNA, 6 bp, artificial
  Tag region
ccgccc

SEQ ID NO: 73 - DNA, 4 bp, artificial
  Tag region
cccg

SEQ ID NO: 74 - DNA, 5 bp, artificial
  Tag region
ggccg

SEQ ID NO: 75 - DNA, 9 bp, artificial
  Tag region
ggggcgggg

SEQ ID NO: 76 - DNA, 23 bp, artificial
  Oligonucleotide for amplifying SNP in bovine calpastin gene
ggggatgactacgagtatgactg

SEQ ID NO: 77 - DNA, 23 bp, artificial
  Oligonucleotide for amplifying SNP in bovine calpastatin gene
gtgaaaatcttgtggaggctgta

SEQ ID NO: 78 - DNA, 20 bp, artificial
  TSP primer L1
cgaggattggctcaagacgc

SEQ ID NO: 79 - DNA, 21 bp, artificial
  TSP primer L2
gcagcgttcttaggactggca

SEQ ID NO: 80 - DNA, 21 bp, artificial
  TSP primer A1
cgaatggattcttcagaaaag

SEQ ID NO: 81 - DNA, 17 bp, artificial
  TSP primer A2
gcgttcctctgcccttg

SEQ ID NO: 82 - DNA, 20 bp, artificial
  TSP primer L1 for fructose-6-phosphate 2-kinase
gcgtcgcaaagacaagctga

SEQ ID NO: 83 - DNA, 20 bp, artificial
  TSP primer L2 for fructose-6-phosphate 2-kinase
ccgcaggcgaacctttacat

SEQ ID NO: 84 - DNA, 19 bp, artificial
  TSP primer A1 for fructose-6-phosphate 2-kinase
cgtgcatactgcacaaaat

SEQ ID NO: 85 - DNA, 21 bp, artificial
  TSP primer A2 for fructose-6-phosphate 2-kinase
gcacctcataaagaatggttc

SEQ ID NO: 86 - DNA, 20 bp, artificial
  TSP primer L1 for unnamed rice gene
gaagtcgacgctgatggcaa

SEQ ID NO: 87 - DNA, 20 bp, artificial
  TSP primer L2 for unnamed rice gene
tcgtgcgatccgttttagca

SEQ ID NO: 88 - DNA, 17 bp, artificial
  TSP primer A1 for unnamed rice gene
gggtcttcggagcacga

SEQ ID NO: 89 - DNA, 17 bp, artificial
  TSP primer A1 for unnamed rice gene
gcaatctcggcgagaag

SEQ ID NO: 90 - DNA, 20 bp, artificial
  TSP primer L1 for gene encoding cytosolic aldehyde dehydrogenase
cggagatcctttcaacccga

SEQ ID NO: 91 - DNA, 20 bp, artificial
  TSP primer L2 for gene encoding cytosolic aldehyde dehydrogenase
tcggatgtccgtccagatca

SEQ ID NO: 92 - DNA, 21 bp, artificial
  TSP primer A1 for gene encoding cytosolic aldehyde dehydrogenase
ggcattttgtaacatgttcag

SEQ ID NO: 93 - DNA, 18 bp, artificial
  TSP primer A2 for gene encoding cytosolic aldehyde dehydrogenase
cggtcggtaagagcgaag

SEQ ID NO: 94 - DNA, 48 bp, artificial
  sequence flanking 677C/T allele in human MTHFR gene
tgaaggagaaggtgtctgcgggagycgatttcatcatcacgcagcttt

SEQ ID NO: 95 - DNA, 20 bp, artificial
  TSP primer F-LS.MTHFR for MTHFR gene
tcttcatccctcgccttgaa

SEQ ID NO: 96 - DNA, 20 bp, artificial
  TSP primer R-LS.MTHFR
gcctgccgttttctcctctt

SEQ ID NO: 97 - DNA, 16 bp, artificial
  TSP primer F-AS.MTHFR_REF
gcgtgtctgcgggagc

SEQ ID NO: 98 - DNA, 17 bp, artificial
  TSP primer R-AS.MTHFR
cggatggggcaagtgat

SEQ ID NO: 99 - DNA, 20 bp, artificial
  TSP primer F-LS.HSV for HSV-1 and HSV-2 virion glycoprotein B gene
gccaccgctactcccagttt

SEQ ID NO: 100 - DNA, 19 bp, artificial
  TSP primer R-LS.HSV for HSV-1 and HSV-2 virion glycoprotein B gene
cctcctcgacgatgcagtt

SEQ ID NO: 101 - DNA, 18 bp, artificial
  TSP primer F-AS.HSV-1 allele-specific for HSV-1 glycoprotein B gene
cacgacatggagctgaaa

SEQ ID NO: 102 - DNA, 22 bp, artificial
  TSP primer F-LS.HSV for HSV-1 and HSV-2 virion glycoprotein B gene
cagcgccatgtcaacgatatgt

SEQ ID NO: 103 - DNA, 20 bp, artificial
  TSP primer R-LS.HSV for HSV-1 and HSV-2 virion glycoprotein B gene
cgcatcgagttttggacgat

SEQ ID NO: 104 - DNA, 14 bp, artificial
  TSP primer F-AS.HSV(MP-S) allele-specific for HSV-1 glycoprotein B gene
tgcatcgcctcggc

SEQ ID NO: 105 - DNA, 22 bp, artificial
  TSP primer F-LS.SA for bacterial 16S rRNA gene
tggagcatgtggtttaattcga

SEQ ID NO: 106 - DNA, 19 bp, artificial
  TSP primer R-LS.SA for bacterial 16S rRNA gene
tgcgggacttaacccaaca

SEQ ID NO: 107 - DNA, 20 bp, artificial
  TSP primer F-AS.SA allele-specific for Staphylococcus aureus 16S rRNA gene
cgcttaccaaatcttgacat

(19) United States
(12) Patent Application Publication
(10) Pub. No.: US 2010/0297633 A1
(43) Pub. Date: Nov. 25, 2010

(54) METHOD OF AMPLIFYING NUCLEIC ACID

(75) Inventors: Matthew James Hayden, Victoria (AU); Tania Tabone, Victoria (AU)

Correspondence Address: KNOBBE MARTENS OLSON & BEAR LLP, 2040 MAIN STREET, FOURTEENTH FLOOR, IRVINE, CA, 92614, US

(73) Assignee: MOLECULAR PLANT BREEDING NOMINEES LTD, Bundoora (AU)

(21) Appl. No.: 12/678,874

(22) PCT Filed: Sep. 19, 2008
(86) PCT No.: PCT/AU2008/001396

§ 371 Date: Aug. 13, 2010

Related U.S. Application Data
(60) Provisional application No. 60/973,928, filed on Sep. 20, 2007.

(51) Int. Cl. C12Q 1/68; G01N 33/559; G01N 33/50

(52) U.S. Cl. 435/6; 204/450; 204/456; 204/451

(57) ABSTRACT

The present invention provides a method for detecting a polymorphism or mutation in nucleic acid comprising a first phase to amplify or enrich for a sequence comprising a polymorphism or mutation and a second phase for detecting the polymorphism or mutation, wherein both phases are performed in the same reaction vessel.

### CROSS-REFERENCE TO RELATED APPLICATION

The present application claims priority from U.S. Ser. No. 60/973,928 filed in the United States Patent and Trademark Office on Sep. 20, 2007, the contents of which are incorporated by reference in their entirety.

### FIELD OF INVENTION

The present invention relates to methods for detecting a polymorphism or a mutation, such as by polymerase chain reaction (PCR), and applications therefor.

### BACKGROUND OF INVENTION

### Description of Related Art

Genetic variations between organisms, such as polymorphisms and mutations are detected in a variety of assays used in, for example, gene mapping, positional cloning, identification of individuals (e.g., for animal or plant marker-assisted breeding or for forensic identification, maternity testing, paternity testing), genotype/phenotype association, for determining a subject likely to develop a trait of interest or for determining a subject at risk of developing a genetic disorder.

Single nucleotide polymorphisms (SNPs) are the most common type of genetic variation within the genome of several organisms. For example, a SNP occurs on average once per 250-1000 base pairs (bp) and account for 90% of sequence variants in the human genome (Collins et al., Genome Res., 8: 1229-1231, 1998). As for plants, in maize a SNP occurs on average once every 104 base pairs (Tenaillon et al., Proc. Natl. Acad. Sci. USA, 98: 9161-9166); soybean has about one SNP every 273 bp (Zhu et al., Genetics, 163: 1123-1134, 2003); wheat has one SNP about every 200 bp (Ravel et al., In: Vollman et al (Eds) Genetic variation for plant breeding, Eucarpia: Tulln, Austria, pp 177-181); and rapeseed has one SNP about every 600 bp (Fourmann et al., Theor. Appl. Genet. 105: 1196-1206). The high density and mutational stability of SNPs make them particularly useful genetic markers for population genetics and for mapping genes associated with complex traits.

Nucleic acid amplification techniques have become key tools for detecting nucleotide sequence variations. Several of the most common techniques currently used for amplification and analysis of genetic markers use a polymerase (such as, for example, a DNA polymerase and/or a RNA polymerase) to replicate a nucleic acid template using, for example, a polymerase chain reaction (PCR; e.g., Saiki et al., Science 230:1350, 1985). These methods are useful for amplifying nucleic acid from DNA, DNA/RNA hybrid or RNA and/or for determining the nucleotide sequence of a specific nucleic acid (e.g., by sequencing, allele specific PCR or primer extension). Generally, a polymerase-mediated replication technique uses a primer (e.g., a short oligonucleotide) capable of annealing selectively to a nucleic acid template to provide the binding site for the polymerase to initiate replication. By iteratively annealing the primer and replicating the nucleic acid template of interest, the nucleic acid is amplified.

A standard PCR is performed using two oligonucleotide primers designed to hybridize to opposite strands of a double stranded nucleic acid adjacent to the region of interest. Strands of nucleic acid in a sample are separated, typically by thermal denaturation, and the primers then allowed to anneal to the single strand templates. These primers provide the site of binding for a polymerase and initiate replication of the region of interest. Both the original nucleic acid and the newly synthesized nucleic acid are then be used as templates for further amplification cycles, thereby permitting exponential amplification of the nucleic acid region of interest.

An example of a method for detecting a polymorphism using PCR is the PCR-restriction fragment length polymorphism (RFLP) method, involving a combination of the polymerase chain reaction (PCR) method and cleavage with restriction enzymes (Olerup, Tissue Antigens, 36:83-87, 1990). In this method a nucleic acid comprising a polymorphism an allele of which modifies the binding site of a restriction endonuclease is amplified by PCR, and the resulting amplification product contacted with the restriction endonuclease under conditions sufficient for cleavage to occur in the presence of the correct binding site. By resolving the resulting nucleic acid fragments, e.g., using electrophoresis, the presence or absence of the restriction endonuclease binding site is determined, as is the sequence of the allele. However, this method is time consuming, because it requires both a PCR and treatment with a restriction enzyme for a sufficient time for cleavage to occur (typically, 3 to 24 hours).

Another method used for detection of a polymorphism is a single-strand conformation polymorphism (SSCP) detection method. SSCP detection is based on the principle that single-strand DNA and RNA having different sequences exhibit different electrophoretic mobility in polyacrylamide gels. This method involves amplifying a sequence comprising a polymorphism using PCR and separating the resulting amplicons to thereby determine their electrophoretic mobility and, as a consequence, the sequence of the polymorphism. However, SSCP methods require that the experimental conditions are strictly controlled to detect subtle differences in electrophoretic mobility. Accordingly, the methods are extremely complicated. Moreover, such a method is not readily adapted to detection of polymorphisms in a polyploid organism or a specific gene in a well-conserved gene family. This is because, the method relies on amplification of short nucleic acid fragments (i.e., less than about 300 bp). The size restrictions of SSCP methods mean that it may not be possible to selectively amplify a fragment comprising the polymorphism to be detected, e.g., a fragment from a homologous gene or homologous genes in a polyploid organism may also be amplified, thereby confounding the results of the analysis.

Assays such as TaqMan® and Molecular Beacon® assays, have also been produced which amplify a sequence comprising a polymorphism using PCR and detecting an allele of the polymorphism with an oligonucleotide probe that selectively binds to one allele of the polymorphism. The probe is labeled with a fluorescent moiety and a quencher moiety. In the absence of binding to the allele, the quencher moiety prevents the fluorescent moiety from emitting a detectable signal. When bound to the allele, the fluorescent moiety and quencher moiety are separated, and the fluorescent moiety emits a detectable signal. A disadvantage of TaqMan® and Molecular Beacon® assays is that they are expensive since they require specialized probes to detect a polymorphism. These assays are also not readily adapted to detection of polymorphisms in polyploid species, e.g., wheat or in well conserved gene families (Giancola et al., Theor. Appl. Genet., 112: 1115-1124, 2006). This is because the assays require amplification of relatively short sequences, e.g., about 150 bp, prior to detection. Accordingly, the methods may not be amenable to amplifying a sequence specific to the nucleic acid comprising the polymorphism of interest, e.g., they may amplify related genes in a gene family and/or homologous genes in a polyploid organism.

Previous methods for detecting polymorphisms in polyploid organisms, such as plants have generally involved amplifying a sequence from one genome comprising a polymorphism of interest, isolating the amplification product and detecting the polymorphism in the amplification product. Accordingly, these methods are often complex, requiring multiple steps to amplify and isolate a nucleic acid specific to one genome from the polyploid organism. Moreover, the requirement for multiple steps, often requiring additional handling of a sample increases the risk of contamination of a sample.

It will be apparent form the foregoing that notwithstanding the advances in methods for detecting polymorphisms, it is clear that these methods suffer from several disadvantages, such as, for example, lengthy assay time, increased expense, complicated assay format and inability to detect polymorphisms in genes from conserved gene families or in polyploid organisms. Accordingly, it is clear that there is a need in the art for a rapid and inexpensive assay that enables detection of a polymorphism in a sample, including a sample from a polyploid organism. Such an assay would have clear utility in, for example, diagnosis of a condition and/or the identification of an individual or group thereof.

Conventional techniques of molecular biology and recombinant DNA technology used in performance of the present invention are described, for example, in the following texts that are incorporated by reference:
i. Sambrook, Fritsch & Maniatis, Molecular Cloning: A Laboratory Manual, Cold Spring Harbor Laboratories, New York, Second Edition (1989), whole of Vols I, II, and III;
ii. DNA Cloning: A Practical Approach, Vols. I and II (D. N. Glover, ed., 1985), IRL Press, Oxford, whole of text;
iii. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
iv. Nucleic Acid Hybridization: A Practical Approach (B. D. Hames & S. J. Higgins, eds., 1985) IRL Press, Oxford, whole of text;
v. Perbal, B., A Practical Guide to Molecular Cloning (1984);
vi. Methods In Enzymology (S. Colowick and N. Kaplan, eds., Academic Press, Inc.), whole of series;

### SUMMARY OF THE INVENTION

### Introduction

In work leading up to the present invention, the present inventors sought to produce a simple and inexpensive method for detecting a polymorphism or mutation, and that was amenable to detecting a polymorphism in a specific gene from a conserved gene family or in nucleic acid from a polyploid organism. The inventors also sought to produce a method for detecting a polymorphism or mutation that does not require multiple distinct reactions, thereby reducing costs and risks of contamination.

As exemplified herein, the inventors have produced a PCR-based method for detecting a polymorphism or mutation comprising multiple phases of amplification, i.e., a first phase to amplify or enrich for a sequence comprising a polymorphism or mutation, and a second phase for detecting the polymorphism or mutation. As exemplified herein, the method developed by the inventors comprises a first amplification phase in which a set of first primers is used to selectively amplify a nucleic acid comprising the polymorphism or mutation, i.e., to enrich for the sequence comprising the polymorphism or mutation. A second phase amplification is performed using one or more second primers comprising (i) an allele specific region comprising a sequence complementary to the template nucleic acid adjacent to the polymorphism or mutation and that has a lower Tm than the first primers; and (ii) a tag region comprising a sequence that does not anneal to the template nucleic acid, however increases the Tm of the second primer to about the Tm of the first primer. By reducing the annealing temperature in the second phase amplification, the allele specific region of the second primer(s) anneals to the amplification product of the first phase amplification, thereby permitting amplification with the second primer(s) and first primers. Following several amplification cycles, the sequence of the second primer(s) is incorporated into amplification products thereby permitting the annealing temperature to be increased, and for the entire second primer and the first primer to anneal to target sequences and prime amplification by PCR. By detecting one or more amplification products produced in this second phase of amplification a polymorphism or mutation is detected.

In one exemplified form of the present invention a second primer comprises one or more nucleotide(s) positioned at the 3′ end of the allele specific region that is complementary to an allele of the polymorphism or mutation. The 3′ end of the second primer only anneals in the presence of that allele, and permits amplification by PCR. Detection of the amplification product produced using this second primer and either another second primer or a first primer, an allele of a polymorphism or mutation is detected. On the other hand, failure to detect an amplification product produced using this primer may indicate the presence of a different allele. Use of two or more second primers complementary to different alleles permits positive detection of different alleles. In this respect, the two or more second primers may be used in the same reaction if each primer is labeled so as to permit differentiation between amplification products produced by different primers, e.g., using tag regions having different molecular weights or different detectable markers. Alternatively, each second primer is used in a separate reaction.

The assay of the present invention can thus be configured utilizing a variety of primer combinations, including one or a plurality of locus-specific primers and one or a plurality of allele-specific primers for allele discrimination, wherein the primers bind to the same or opposite nucleic acid strands and/or are differentially labeled and wherein the products are resolved e.g., on a variety of size separation matrixes e.g., agarose gel, polyacrylamide, or using eGENE by end-point or real-time melting analysis using instrumentation such as the RotorGene6000 (Corbett Research). For example, data presented in example 1 hereof demonstrate allelic discrimination by differential product size using a pair of allele-specific primers designed for opposite DNA strands, to thereby permit codominant allelic discrimination in a single reaction, wherein the sizes of the resulting PCR products are resolved on a variety of size separation matrices e.g., agarose gel, polyacrylamide, or using eGENE by end-point or real-time melting analysis using instrumentation such as the RotorGene6000 (Corbett Research). In another example, data presented in example 2 hereof demonstrate allelic discrimination by differential product size using a pair of allele specific primers designed to the same DNA strand, to thereby permit codominant allelic discrimination using a size separation matrix such as that described in the preceding paragraph. In another example, data presented in example 3 hereof demonstrate allelic discrimination using a single allele-specific primer e.g., AS₁, wherein the number of reactions required for genotype determination is influenced by the size of the PCR fragment amplified by the locus specific (LS) primer pair. In another example, data presented in example 4 hereof demonstrate allelic discrimination by differential product labeling using a pair of allele-specific (AS) primers designed to the same DNA strand, wherein AS primers differ by having a detectable marker, such as a fluorescent dye, attached to their 5′-ends such that differential detection of the detectable marker attached to each AS primer facilitates codominant allelic discrimination. In another example, data presented in example 5 hereof demonstrate allelic discrimination by end point and/or real time high resolution melting analysis using a pair of AS primers designed to anneal to a region adjacent the allele being detected such that they amplify nucleic acid comprising the allele. In another example, data presented in example 6 hereof demonstrate allelic discrimination by end point and/or real time high resolution melting analysis using a single AS primer designed to anneal to a region adjacent the allele being detected such that it amplifies nucleic acid comprising the allele. The present inventors have also demonstrated that, for some assay configurations, such as allelic discrimination by differential product detection e.g., as demonstrated in examples 5 and 6, the capture of sequence variation within the second phase PCR amplification product eliminates the requirement for AS primers to contain mismatched nucleotides that can cause primer annealing destabilization.

As exemplified herein, the method of the present invention is biphasic as demonstrated by real-time polymerase chain reaction (PCR) to monitor the accumulation of the first and second phase products e.g., in assays configured for allelic discrimination wherein differential product sizes are identified for each phase e.g., a first phase employing locus-specific (LS) primers e.g., L₁, L₂, L₃, etc., and a second phase employing allele-specific (AS) primers A₁, A₂, A₃, etc. Data presented in example 8 hereof affirm the reaction mechanism of the assay of the invention i.e., sequential enrichment of a target sequence harboring the SNP by the LS primers L₁ and L₂, followed by nested amplification of the interrogated allele by the AS primers A₁ and A₂.

The inventors have empirically determined parameters for minimizing the participation of AS primers in the first phase of amplification performed using LS primers, as shown in example 7 and example 13 hereof for two model genes, wherein examination of the PCR specificity and yield suggests that AS primers having melting temperatures below about 48° C., and preferably in the range of about 40° C. to about 48° C., and still more preferably having melting temperatures of about 45° C., do not participate significantly in the first phase of amplification. Preferred AS primers should also be selected to be at least about 12-15 nucleotides in length and not exceeding about 36-40 nucleotides in length, preferably having a size range of about 15 nucleotides in length to about 36 nucleotides in length.

Alternatively, or in addition, the annealing efficiency of AS primers is normalized during the initial cycles of the second phase of TSP amplification e.g., by increasing the melting temperature on the complementary region to the AS forward primer, to thereby facilitate correct genotype determination for genomic loci producing mismatched product in samples.

As exemplified herein, the separation of the amplification phases by using different annealing temperatures permits the method produced by the inventors to be performed in a single closed-tube reaction. Accordingly, the method produced by the inventors reduces the risk of contamination caused by sample handling, and is simple since all reagent required for PCR may be included in a single tube.

The inventors have shown that the exemplified method is useful for detecting a polymorphism or mutation in a sample. Such a method is useful for characterizing or identifying one or more individuals, isolates of an organism, cultivars of an organism, species or genera, e.g., based on one or more polymorphisms or mutations in the genome of said individuals, isolates of an organism, cultivars of an organism, species or genera. The method of the present invention can also be applied to identifying a subject having a trait or a disease or having a predisposition to developing a trait or disease, e.g., for marker assisted breeding.

In one example, e.g., example 12 hereof, the inventors have demonstrated sensitivity and accuracy of the assay of the invention for actual genotype determination in plants, wherein mapping populations, each comprising about 250 individuals, were screened independently for SNPs on chromosome 2H containing a frost tolerance QTL and on chromosome 5H containing a malting quality QTL, using cleaved amplified polymorphism (CAP) assays (Minamiyama et al., Plant Breeding 124: 288-291, 2005) and the assay of the present invention, and which demonstrates concordance between the two genotyping methods across all assays.

The inventors have also demonstrated that the exemplified method is useful for the detection of polymorphisms in polyploid organisms, e.g., wheat. This is because the first amplification phase of the method permits use of one or more primers that selectively anneal to one genome of the polyploid organism comprising a polymorphism or mutation to thereby enrich for that sequence prior to detection of the polymorphism or mutation in the second phase of amplification.

In another example, e.g., example 14 hereof, the inventors have demonstrated efficacy of the method of the invention for discrimination of alleles comprising cytosine or thymine at position 677 of the coding region of a gene encoding methylenetetrahydrofolate reductase (MTHFR) of humans. A C677T mutation results in substitution of valine for alanine at position 222 of the encoded protein, thereby producing a thermolabile protein associated with folic acid deficiency, neural tube defects, arterial and venous thrombosis, cardiovascular disease and schizophrenia. Homozygotes carrying two 677T alleles have decreased risk of developing leukemia and/or colon cancer.

In another example, e.g., example 15 hereof, the inventors provide means for using the method of the present invention for detecting HSV-1 and HSV-2 in samples, and for discriminating between HSV-1 and HSV-2. In yet another example, e.g., example 16 hereof, the inventors provide means for using the method of the present invention for detecting HSV-1 in samples, and for discriminating between strains or isolates of HSV-1. In yet another example e.g., example 17 hereof, the inventors provide means for discriminating between Staphylococcus aureus and other bacteria. These examples demonstrate the broad applicability of the invention to clinical diagnoses of disease and infectious agents in animals and humans, and more particularly, for detecting pathogens such as pathogenic bacteria, viruses, fungi, protists, protozoa or parasites, in samples taken from subjects suspected of being infected. The present invention is clearly useful for detection of infection in early stages, such as before the onset of disease symptoms, for detecting outbreaks of infectious disease such as epidemics or pandemics, and more particularly, for discrimination between pathogenic strains of an organism, the identification of new strains of pathogenic organisms, and for the identification of non-cultivatable or slow-growing microorganisms such as mycobacteria, anaerobic bacteria and viruses. The biphasic reaction mechanism of the present invention permits PCR specificity to be introduced during the first and/or second phase of amplification. The biphasic TSP assay mechanism can be especially useful for diagnostic tests, since it allows for both the detection of the presence-absence of an infectious agent, as well as the identification of the particular species, ecotype, serotype or strain of an infectious agent in a single assay.

### Specific Embodiments

The scope of the invention will be apparent from the claims as filed with the application that follow the examples. The claims as filed with the application are hereby incorporated into the description. The scope of the invention will also be apparent from the following description of specific embodiments.

In one example, the present invention provides a method for detecting a polymorphism or mutation in nucleic acid, said method comprising:

(i) performing a polymerase chain reaction (PCR) under conditions sufficient to amplify a nucleic acid template comprising a polymorphism or mutation with one or more set(s) of first primers thereby producing a first amplification product, said set(s) of first primers capable of annealing selectively to a nucleic acid template comprising a polymorphism or mutation at a first temperature;
(ii) performing PCR under conditions sufficient to amplify the first amplification product with one or more second primer(s) or set(s) of second primers and/or with one or more of the primers from the set of first primers thereby producing a second amplification product comprising a sequence complementary to the allele-specific region and the tag region, said second primer(s) comprising an allele-specific region capable to annealing to the nucleic acid template and/or the first amplification product and a tag-region that does not anneal to the nucleic acid template, wherein said allele-specific region has a melting temperature (Tm) lower than the first primer and is not capable of annealing selectively to the template nucleic acid or the first amplification product at the first temperature and wherein the second primer is capable of annealing selectively to a nucleic acid comprising a sequence complementary to the allele-specific region and the tag region at similar to the first temperature, wherein said conditions comprise an annealing temperature suitable for annealing of the allele-specific region of the second primer(s) or set(s) of second primers to the first amplification product and/or the template nucleic acid and for the annealing of the first set of primers to the first amplification product and/or the template nucleic acid;
(iii) performing PCR under conditions sufficient to amplify the second amplification product to produce one or more third amplification product(s), said conditions comprising an annealing temperature suitable for annealing of the second primer(s) or set(s) of second primers to the second amplification product and for annealing of one or more primers from the set of first primers to the second amplification product but not for annealing of the allele specific region of the second primer(s) or set(s) of second primers to anneal selectively to the first amplification product at a detectable level, wherein the third amplification product(s) is/are amplified with the set(s) of second primers and/or a second primer and a first primer; and
(iv) detecting the third amplification product(s) with a detection means, wherein detection of said third amplification product(s) is/are indicative of the polymorphism or mutation.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “polymorphism” shall be taken to mean a naturally-occurring variation in the nucleotide sequence of a specific site or region of the genome of a subject, or an expression product thereof that occurs in a population of subjects. Preferably, the polymorphism is a single nucleotide polymorphism (SNP).

As used herein, the term “mutation” shall be taken to mean a permanent, transmissible change in nucleotide sequence of the genome of a subject and optionally, an expression product thereof. Examples of mutations include an insertion of one or more new nucleotides or deletion of one or more nucleotides or substitute of one or more existing nucleotides with different nucleotides.

As used herein, the term “PCR” or “polymerase chain reaction” shall be taken to mean an amplification reaction employing multiple cycles of (i) denaturation of double-stranded nucleic acid such as a nucleic acid template to be amplified or a hybrid between a template and a complementary primer; (ii) annealing of a primer to its complementary sequence in the single-stranded “template”; and (iii) extension of the primer in the 5′- to 3′-direction by a polymerase activity e.g., an activity of a thermostable polymerase, such as, Taq, to thereby produce a double-stranded nucleic acid comprising a newly-synthesized strand complementary to the single-stranded template. By utilizing two primers capable of annealing to the complementary strands in the double-stranded template (i.e., to each denatured single-stranded template), multiple copies of the template are produced in each cycle, thereby amplifying the template. Many formats of PCR are known in the art including, for example, reverse-transcriptase mediated PCR(RT-PCR), nested PCR, touch-up and loop incorporated primers (TULIP) PCR, touch-down PCR, competitive PCR, rapid competitive PCR (RC-PCR), and multiplex PCR.

As used herein the term “template nucleic acid” includes DNA, RNA or RNA/DNA with or without any nucleotide analogs therein including single-stranded or double-stranded genomic DNA, mRNA or cDNA. The present invention is not limited by the nature or source of the template nucleic acid. The template nucleic acid can be derived directly or indirectly from an organism, a tissue or cellular sample obtained previously from an organism, or can be present in an aqueous or non-aqueous extract of a tissue or cellular sample.

As will be known to the skilled artisan, a “primer” is a nucleic acid molecule comprising any combination of ribonucleotides, deoxyribonucleotides and analogs thereof such that it comprises DNA, RNA or DNA/RNA, optionally with one or more ribonucleotide or deoxyribonucleotide analogs contained therein, and capable of annealing to a nucleic acid template to act as a binding site for an enzyme, e.g., DNA or RNA polymerase, thereby providing a site for initiation of replication of a specific nucleic acid in the 5′ to 3′ direction. The nucleotide sequence of a primer is generally substantially complementary to the nucleotide sequence of a template nucleic acid to be amplified, or at least comprises a region of complementarity sufficient for annealing to occur and extension in the 5′ to 3′ direction therefrom. However, as will be apparent to the skilled artisan a degree of non-complementarity will not significantly adversely affect the ability of a primer to initiate extension. Suitable methods for designing and/or producing a primer suitable for use in the method of the present invention are known in the art and/or described herein. Primers are generally, but not necessarily, short synthetic nucleic acids of about 12-50 nucleotides in length. Preferably, each primer of the set of first primers and/or the allele-specific region of the second primer(s) or each primer of the set of second piimer(s) comprises at least about 12-30 nucleotides in length capable of annealing to a strand of the nucleic acid template.

The term “set” with reference to a “set of first primers” or a “set of second primers” or more generally to a “set of primers” shall be taken to mean a number of primers having different, albeit not necessarily entirely different, sequences. A preferred set of primers will comprise primers that are capable of annealing to opposite DNA strands and priming the amplification of an amplification product from one or more template molecules.

By “amplification product” is meant an amplified sequence, which may be nucleic acid comprising a polymorphism or mutation.

In the present context, the term “annealing” or similar term shall be taken to mean that a primer and a nucleic acid to be amplified (i.e., template or amplification product) are base-paired to each other to form a double-stranded or partially double-stranded nucleic acid, using a temperature or other reaction condition known in the art to promote or permit base-pairing between complementary nucleotide residues. As will be known to the skilled artisan, the ability to form a duplex and/or the stability of a formed duplex depends on one or more factors including the length of a region of complementarity between the primer and nucleic acid to be amplified, the percentage content of adenine and thymine in a region of complementarity (i.e., “A+T content”), the incubation temperature relative to the melting temperature (Tm) of a duplex, and the salt concentration of a buffer or other solution in which the amplification is performed. Generally, to promote annealing, the primers and nucleic acid to be amplified are incubated at a temperature that is at least about 1-5° C. below a primer Tm that is predicted from its A+T content and length. Duplex formation can also be enhanced or stabilized by increasing the amount of a salt (e.g., NaCl, MgCl₂, KCl, sodium citrate, etc) in the reaction buffer, or by increasing the time period of the incubation, as described by Sambrook et al., Molecular Cloning: A Laboratory Manual, Cold Spring Harbor Laboratory Press; Hames and Higgins, Nucleic Acid Hybridization: A Practical Approach, IRL Press, Oxford (1985); Berger and Kimmel, Guide to Molecular Cloning Techniques, In: Methods in Enzymology, Vol 152, Academic Press, San Diego Calif. (1987); or Ausubel et al., Current Protocols in Molecular Biology, Wiley Interscience, ISBN 047150338 (1992).

As used herein, the term “anneals selectively” shall be taken to mean that a primer anneals to a target nucleic acid, e.g., a template nucleic acid and/or an amplification product more often than it anneals to another nucleic acid, e.g., to produce a signal that is significantly above background (i.e., a high signal-to-noise ratio). The level of specificity of annealing is determined, for example, by performing an amplification reaction using the primer and detecting the number of different amplification products produced. By “different amplification products” is meant that amplified nucleic acids of differing nucleotide sequence and/or molecular weight to the target nucleic acid. Clearly, amplification products that differ in molecular weight are readily identified, for example, using gel electrophoresis. A primer that selectively anneals to a target nucleic acid produces an amplification product from that target nucleic acid at a level greater than any other amplification product.

The absence of detectable annealing of the allele-specific region of the second primer or set of second primers to the template and/or the first amplification product is determined empirically e.g., by the appearance of a correct amplification product following a first round amplification or alternatively by the absence of detectable amplification of template using a second primer or set thereof at the first temperature. This selectivity is partially attributed to the fact that the first primer or set thereof has a greater predicted melting temperature (Tm) than the allele-specific region of the second primer or set thereof. Preferably, the first primer or set thereof has a Tm at least about 10° C. to about 21° C. greater than that of the second primer or set thereof. More preferably, the first primer or set thereof has a Tm at least about 12° C. or about 15° C. or about 18° C. or about 21° C. greater than that of the second primer or set thereof. Methods for determining the Tm of a primer are known in the art and/or described herein.

In one example, the Tm of the first primer is between about 60° C. and about 75° C., more preferably between about 60° C. and about 65° C., even more preferably about 61° C. or 62° C. or 63° C. or 64° C. or 65° C. It is also preferred for first primers i.e., locus-specific primers to amplify a region in nucleic acid having a length greater than about 400 bp in length, preferably greater than about 450 bp or about 500 bp in length. Although not essential to the present invention, it is preferred for the locus-specific primer to comprise a 3′-nucleotide complementary to a SNP allele present at the locus of interest.

The second primer comprises an allele-specific region and a tag. In one example, the Tm of the allele specific region of the second primer is between about 35° C. and about 50° C., more preferably between about 42° C. and about 48° C., even more preferably about 42° C. or 43° C. or 44° C. or 45° C. or 46° C. or 47° C. or 48° C. Allele specific regions of the second primers are preferably between about 12 nucleotides and about 40 nucleotides in length, preferably between about 15 nucleotides in length and about 36 nucleotides in length, including about 18 nucleotides in length or about 21 nucleotides in length or about 24 nucleotides in length or about 27 nucleotides in length or about 30 nucleotides in length or about 33 nucleotides in length or about 36 nucleotides in length. It is also preferred for allele-specific regions of second primers to amplify a region in nucleic acid having a length between about 90 bp and about 300 bp in length, preferably between about 100 bp and about 250 bp in length or between about 100 bp and about 200 bp in length or between about 100 bp and about 150 bp in length.

The second primer should include a tag region at the 5′ end thereof that is not complementary to the target DNA, and preferably increases the melting temperature of the primer relative to a second primer comprising an allele specific region and lacking a tag by about 5° C. or 6° C. or 7° C. or 8° C. or 9° C. or 10° C. or 11° C. This means that the melting temperature of a second primer comprising an allele specific region and a tag is between about 40° C. and about 59° C., more preferably between about 47° C. and about 59° C., even more preferably about 47° C. or 48° C. or 49° C. or 50° C. or 51° C. or 52° C. or 53° C. or 54° C. or 55° C. or 56° C. or 57° C. or 58° C. or 59° C.

An alternative technique to determine the selective annealing of a primer of the invention comprises performing a search of known nucleotide sequences from the sample being assayed (e.g., a database of known sequences from an organism or cell from which the template nucleic acid is derived). Using this technique a sequence similar to or complementary to the sequence of the primer is identified. Whilst such a technique does not ensure selective annealing it is useful for determining a primer (or set of primers) capable of annealing to a plurality of sites in a nucleic acid and possibly producing multiple amplification products (i.e., non-selective annealing).

In one example of the invention, the first primer is a “locus-specific primer”. As used herein, a locus-specific primer is a primer that binds to one or more closely related loci i.e., one or more members of a gene family, one or more homologous genes or parologous genes, etc. For example, locus-specific primers selectively anneal to a template nucleic acid in a sample comprising a plurality of related nucleotide sequences.

In one example, the second primer is “allele-specific” i.e., able to distinguish between one or more related sequences amplified using locus-specific primer(s), by virtue of comprising an allele-specific region. In one example, an allele specific region anneals to a region of a template nucleic acid and/or a region of a first amplification product comprising a polymorphism or mutation. For example, the allele-specific region comprises a nucleotide complementary to the sequence of an allele of a polymorphism or mutation. Preferably, a nucleotide complementary to the sequence of an allele of a polymorphism or mutation is positioned at the 3′ end of the allele specific region, e.g., to facilitate amplification by PCR when said nucleotide anneals to said allele. In an alternative example, the allele-specific region comprises a sequence complementary to a sequence adjacent to a polymorphism or mutation.

By “tag region” is meant a region of a second primer other than an allele-specific sequence that does not anneal to the template nucleic acid or first amplification product. The tag region also comprises a sequence that, in combination with the sequence of the allele-specific region has a Tm similar to the Tm of the first primer. For example, the Tm of the allele specific region and the tag region combined is within about 4° C. or 5° C. or 6° C. or 7° C. of the Tm of the first primer. In one example, the tag region is at least about 4 nucleotides in length, for example about 5 nucleotides in length, preferably at least about 6 nucleotides in length, more preferably at least about 7 nucleotides in length and still more preferably at least about 8 nucleotides in length. It will be apparent to the skilled artisan from the description herein that amplification from PCR is initiated from the 3′ nucleotide of the allele-specific region of the second primer. Proceeding on this basis, the tag region is located 5′ to the allele-specific region in the second primer.

In one example, amplification of a first and a second and a third amplification products is performed in a single reaction vessel, and reagents suitable for performing PCR are provided in said reaction vessel, said reagents comprising the first primer or set of first primers and said second primer or set of second primers.

The term “reaction vessel” shall be construed in its broadest context to include any standard vessel suitable for performing a PCR, such as, for example, a reaction tube (such as, for example, an Eppendorf tube, a polypropylene tube, a glass tube or a glass/plastic composite tube), capillary, microtitre well, or a solid substrate such as a glass slide, microarray matrix, or tissue slice.

The term “providing in a reaction vessel” shall be taken to include the supply of one or more reaction vessels with reagents therein, or alternatively, the provision of a reaction vessel with any number of reagents therein, and separately one or more reagents, with instructions for their combination. Preferably, at least the primers are provided in a reaction vessel, or alternatively, provided separately with instructions for their combination.

The skilled artisan will be aware of reagents suitable for performing PCR, such as, for example, primers, template nucleic acid, ribonucleotide triphosphates and/or deoxyribonucleotide triphosphates or analogs thereof, an appropriate reaction buffer, and a polymerase enzyme (e.g., a thermostable polymerase). Other reaction components known to the skilled artisan are not excluded.

Preferably, no additional components are added to the reaction vessel after amplification of the template has commenced and the reaction volume is not modified by the addition or subtraction of any reagents after this point. This feature of the invention avoids or reduces contamination problems associated with excessive sample handling.

As discussed herein above, one example of the method of the present invention makes use of one or more second primer(s) comprising one or more 3′ terminal nucleotide(s) of the allele-specific region complementary to an allele of a polymorphism or mutation, wherein said primer(s) detectably produce the second amplification product and third amplification product only when said 3′ nucleotides anneal to the allele of said polymorphism or mutation. Accordingly, in the presence of an allele complementary to the 3′ nucleotide of the allele-specific region an amplification product is produced. However, in the presence of an allele that is not complementary to the 3′ nucleotide of the allele specific region, an amplification product is not detectably produced.

In one example, the second primer(s) additionally comprise a nucleotide positioned at the second or third nucleotide position from the 3′ terminus of the allele-specific region that is non-complementary to the sequence of the template nucleic acid and the first amplification product. Such a mismatch destabilizes annealing of the 3′ end of the allele specific region, thereby reducing the likelihood that amplification will be initiated from the 3′ end of the allele-specific region when the 3′ nucleotide of the allele-specific region is not complementary to the allele of the polymorphism or mutation.

In one example of the invention, the third amplification product is produced by PCR with a first primer and a second primer. Such a result indicates the presence of an allele of a polymorphism or mutation comprising a sequence complementary to the 3′ end of the allele-specific region of the second primer. Such a method is useful for detecting the presence of an allele of a polymorphism or mutation using only a single second primer, since amplification with this primer also makes use of a first primer already present in the reaction.

In one example, the method of the present invention is performed with an additional second primer can be included in the reaction, wherein said additional second primer anneals to a sequence adjacent to the polymorphism or mutation. In accordance with this example, the method of the invention is performed with a set of second primers, said set of second primers comprising (i) a second primer comprising one or more 3′ terminal nucleotide(s) of the allele-specific region complementary to an allele of said polymorphism or mutation, wherein said primer only detectably produces the second amplification product and the third amplification product when said 3′ nucleotides anneal to the allele of said polymorphism or mutation; and (ii) a second primer that anneals to nucleic acid adjacent to the polymorphism or mutation. In such a situation, detection of an amplification product produced with both second primers is indicative of an allele of a polymorphism or mutation comprising a sequence complementary to the 3′ end of the allele-specific region of the second primer.

On the other hand, if the 3′ terminal nucleotide(s) of the second primer at (i) do(es) not anneal(s) to the allele, a third amplification product is produced by PCR with the second primer at (ii) and a first primer, thereby indicating an allele of the polymorphism or mutation comprising a sequence non-complementary to the sequence of the 3′ nucleotide of the allele specific region of the second primer at (i).

In a further example, a method as described herein according to any embodiment is performed with a plurality of second primers, wherein individual primers in said plurality comprise one or more 3′ nucleotide(s) complementary to a different allele of the polymorphism or mutation wherein said primers only detectably produce a second amplification product and third amplification product when said 3′ nucleotides anneal to the allele of said polymorphism or mutation, and wherein primers having different 3′ complementary nucleotide(s) also comprise a tag region having different molecular weights. In accordance with this example of the present invention, detecting the molecular weight of the third amplification product indicates which second primer has been incorporated into the third amplification product and, as a consequence, the allele of the polymorphism or mutation.

In one example of the method as described herein according to any embodiment, the detection means comprises performing electrophoresis. The skilled artisan will be aware of methods of electrophoresis, such as, for example, polyacrylamide gel electrophoresis or capillary electrophoresis.

In another example of the method as described herein according to any embodiment, the detection means detects the melting temperature of the third amplification product. Examples of such detection means include for example, a LightCycler® (Perkin Elmer). In one example, melting temperature of a nucleic acid is determined by contacting a nucleic acid with a compound that binds to double stranded nucleic acid and emits light, e.g., fluoresces when excited with light of a particular wavelength. The temperature of the nucleic acid is increased, and the temperature at which the amount of fluorescence detected is reduced as a result of the double stranded nucleic acid denaturing into single stranded nucleic acid is considered the melting temperature of the nucleic acid.

In a further example, a method as described herein according to any embodiment is performed with a plurality of second primers, wherein individual primers in said plurality comprise one or more 3′ nucleotide(s) complementary to a different allele of the polymorphism or mutation wherein said primers only detectably produce the second amplification product and the third amplification product when said 3′ nucleotides anneal to the allele of said polymorphism or mutation, and wherein primers comprising different 3′ nucleotide(s) also comprise a different detectable marker. Preferably, the detectable marker is a fluorescent marker, such as, a fluorescent dye, for example, 6-carboxyfluorescein (FAM), VIC, 2,7′,8′-benzo-5′-fluoro-2′,4,7-trichloro-5-carboxyfluorescein (NED) or tetrachloro-6-carboxyfluorescein (TET). In accordance with this embodiment, detection of the detectable marker indicates which second primer has been incorporated into the third amplification product and, as a consequence, the allele of the polymorphism or mutation.

The present invention also provides a method in which the second primer(s) or set(s) of second primers comprise an allele-specific region capable to annealing to nucleic acid adjacent to the polymorphism or mutation. In accordance with this embodiment, neither primer anneals to the site of the polymorphism or mutation. Rather, the polymorphism or mutation is contained within the third amplification product. The polymorphism or mutation is then detected by determining the melting temperature of the third amplification product, wherein the melting temperature of the third amplification product is indicative of the polymorphism or mutation. Suitable methods for detecting melting temperature of a nucleic acid are described herein.

In one example, a method as described herein according to any embodiment additionally comprises providing the template nucleic acid. For example, the nucleic acid is in the form of a biological sample.

As discussed herein above, the present invention is useful for detecting a polymorphism in a polyploid organism. Accordingly, in one example of the present invention, the nucleic acid is from a polyploid organism or a sample comprising template nucleic acid is from a polyploid organism. In accordance with this example of the invention, it is preferred that the first set of primers is capable of annealing selectively to a genome of said polyploid organism comprising the polymorphism or mutation.

The present invention also provides a method as described herein according to any embodiment additionally comprising providing or obtaining or producing the first set of primers and/or providing the second primer(s) or set(s) of second primers. For example, the method additionally comprises synthesizing the first set of primers and/or providing the second primer(s) or set(s) of second primers. Methods for designing and producing a primer are described herein.

In one example, the present invention further comprises combining reagents suitable for performing PCR in a reaction vessel. For example, the method of the present invention comprises combining a first set of primers and one or more second primer(s) or set of second primer(s) in a reaction vessel. Additional suitable reagents will be apparent to the skilled artisan based on the description herein and include, for example, ribonucleotide triphosphates and/or deoxyribonucleotide triphosphates or analogs thereof, an appropriate reaction buffer, and a polymerase enzyme (e.g., a thermostable polymerase).

The present invention is not to be limited to the detection of a single polymorphism or mutation in a single reaction. Rather, the present invention also provides a method for detecting a plurality of polymorphisms or a plurality of mutations or one or more polymorphisms and one or more mutations in a single reaction, i.e., a multiplex reaction. In this respect, one or more of the polymorphisms and/or mutations can be amplified in the first amplification product. Alternatively, each polymorphism and/or mutation can be amplified in a separate first amplification product. The skilled artisan will be aware that for such a multiplex method each of the amplification products detected should have a different molecular weight and/or be labeled with a different detectable marker to thereby permit detection of each amplification product. Methods for predicting amplification products having sufficiently different molecular weight to permit detection in a single reaction will be apparent to the skilled artisan and are described, for example, in International Patent Application No. PCT/AU2006/000318 (International Publication No. WO 2006/094360).

The skilled artisan will be aware that a method for detecting one or more polymorphisms and/or mutations is useful for, for example, determining relationships between one or more individuals, isolates of an organism, cultivars of an organism, species or genera. For example, the method of the present invention is used to detect one or more nucleic acids that are polymorphic between two or more individuals, isolates of an organism, cultivars of an organism, species or genera. Accordingly, the present invention additionally provides for characterizing or identifying one or more individuals, isolates of an organism, cultivars of an organism, species or genera said process comprising performing the method as described herein according to any embodiment to detect one or more polymorphisms and/or mutations in nucleic acid from one or more individuals, isolates of an organism, cultivars of an organism, species or genera, wherein the one or more polymorphisms or mutations is(are) characteristic of one or more individuals, isolates of an organism, cultivars of an organism, species or genera.

It will be apparent to the skilled artisan from the description herein that the present invention is useful for typing an organism within or between groups, or for differentiating between individuals or groups (e.g., for identification of a specific plant variety). The skilled artisan will appreciate that the method of the present invention is also applicable to, for example, the analysis of a sample (e.g., a food sample) to identify the presence of a foreign agent (e.g., a genetically modified plant).

The present invention additionally provides a process for detecting one or more polymorphisms and/or mutations associated with a trait, e.g., to select a subject having a trait or having a predisposition to a trait and/or for the purpose of marker-assisted breeding. For example, the present invention provides a process for screening an animal species to identify an animal having or having a predisposition to a trait of interest, e.g., for the purpose of animal husbandry, e.g., for the selection of a desired trait (e.g., marbled beef from cattle, or enhanced milk quality from cattle, enhanced speed or stamina in horses or enhanced meat quality from pigs). The present invention also provides a process for screening a plant species to identify a plant having a trait or having a predisposition to a trait, such as increased productivity, e.g., resistance to drought, resistance to a disease or a pest, resistance to pre-harvest sprouting, resistance to frost and an increased nutritional quality. In accordance with this embodiment, the present invention provides a process for identifying a subject having a trait or having a predisposition to a trait, said method comprising performing a method as described herein according to any embodiment to detect one or more polymorphism(s) and/or mutation(s) associated with a trait or a predisposition to a trait, wherein detection of said polymorphisms and/or mutations is indicative of a subject having the trait or having a predisposition to developing the trait.

As used herein the term “subject” shall be understood to include a bacterium, virus, fungus, protist, plant, non-human animal or human, including any developmental stage of said bacterium, virus, fungus (e.g., endophytic fungi), protist, plant, non-human animal or human. Specific strains of HSV, and/or specific strains or species or races of brewer's yeast e.g., Saccharomyces sp., and/or specific strains or species or races of Escherichia coli, Staphylococcus aureus e.g., multi-resistant S. aureus (MRSA), or Mycobacterium sp. are particularly contemplated herein.

As used herein, the term “associated with” shall be taken to mean that the presence of a specific genetic marker is significantly correlated with a trait of interest in an organism or a population of organisms. Preferably, the presence of the genetic marker is significantly correlated with the presence of the trait of interest in a population of unrelated organisms.

In one example, the method additionally comprises selecting a subject having the trait or a predisposition to a trait, based on the detection of one or more polymorphism(s) and/or mutation(s) associated with a trait or a predisposition to developing a trait. For example, the subject is selected from a population of subjects.

In one example, a method for identifying or selecting a subject as described herein according to any embodiment additionally comprises obtaining or providing a cell or a gamete or other reproductive material or an embryo or a fetus from the selected or identified subject.

In one example, a method of identifying or selecting a subject of the present invention additionally comprises breeding a subject identified or selected by a method described herein in any embodiment. Optionally, such a method of breeding additionally comprises performing a method described herein to identify and/or select an embryo or a fetus or a plantlet or an offspring plant or an offspring non-human animal or an offspring human comprising one or more polymorphism(s) and/or mutation(s) associated with the trait or a predisposition to the trait.

The skilled artisan will also appreciate that the present invention is also useful for identifying a subject having a disease or disorder or a subject at risk of developing a disease or disorder. In this respect, the present invention also provides a process for identifying a subject having a disease or disorder or at risk of developing a disease or disorder, said process comprising performing a method as described herein according to any embodiment to detect one or more polymorphism(s) and/or mutation(s) that are associated with a disease or disorder, wherein detection of said polymorphism(s) and/or mutation(s) indicates that the subject suffers from the disease or disorder or has a predisposition to the disease or disorder.

The skilled artisan will be aware that this example of the invention relates to the diagnosis of a disease or disorder caused by a mutation, e.g., cystic fibrosis or sickle cell anemia or Tay Sachs disease or folic acid deficiency, and/or to determining a subject at risk of developing a disease or disorder that is associated with a mutation or polymorphism, e.g., Parkinson's disease or Alzheimer's disease or neural tube defect or arterial thrombosis or venous thrombosis or cardiovascular disease or schizophrenia or having increased or decreased risk of developing cancer e.g., leukemia and/or colon cancer.

Furthermore, the present invention is applicable to diagnosis of infection by virtue of detecting and/or identifying an infectious agent that causes an infection and/or for discriminating between strains, ecotypes, serotypes or species of an infectious agent. Clearly, this encompasses the diagnosis and/or prognosis of disease caused by the infectious agent. Accordingly, in another example, the present invention provides a process for identifying an infectious agent in a sample and/or for discriminating between infectious agents in a sample, said process comprising performing a method as described herein according to any embodiment on a sample obtained from a subject to thereby detect one or more nucleic acid sequences of one or more infectious agents, wherein detection of said one or more nucleic acid sequences in the sample indicates the presence of an infectious agent in the sample and/or discriminates between infectious agents in the sample.

The present invention also provides a kit comprising:

(i) one or more set(s) of first primers, said set(s) of first primers capable of annealing selectively to a nucleic acid template comprising a polymorphism or mutation at a first temperature;
(ii) one or more second primer(s) or set(s) of second primers, said second primer(s) comprising an allele-specific region capable of hybridizing to the nucleic acid template and a tag-region that does not anneal to the nucleic acid template, wherein said allele-specific region has a melting temperature (Tm) lower than the first primer and is not capable of annealing selectively to the nucleic acid template at the first temperature and wherein the second primer is capable of annealing selectively to a nucleic acid comprising a sequence complementary to the allele-specific region and the tag region at about the first temperature; and
(iii) optionally, instructions for performing the method as described herein according to any embodiment.

Preferably, the set(s) of first primers and the second primer(s) or set(s) of second primers are provided in a reaction vessel suitable for performing polymerase chain reaction (PCR).

The present invention also provides for the use of a kit as described herein in any embodiment in any method of the present invention.

The present invention also provides a method of producing a set of primers, said method comprising:

(i) producing one or more set(s) of first primers, said set(s) of first primers capable of annealing selectively to a nucleic acid template comprising a polymorphism or mutation at a first temperature; and
(ii) producing one or more second primer(s) or set(s) of second primers, said second primer(s) comprising an allele-specific region capable to hybridizing to the nucleic acid template and a tag-region that does not anneal to the nucleic acid template, wherein said allele-specific region has a melting temperature (Tm) lower than the first primer and is not capable of annealing selectively to the nucleic acid template at the first temperature and wherein the second primer is capable of annealing selectively to a nucleic acid comprising a sequence complementary to the allele-specific region and the tag region at about the first temperature.

In one example, the method further comprises analyzing nucleotide sequence data to thereby determine a panel of candidate primers for inclusion in said set.

In another example, the method of the present invention as described according to any embodiment hereof is performed using the panel of primers to thereby determine a panel of first primer(s) and/or second primer(s) that provide discrimination, more preferably optimum discrimination, between alleles in the nucleotide sequence analyzed.

In another example, the method further comprises selecting a panel of first primer(s) and/or second primer(s) that provide discrimination, more preferably optimum discrimination, between alleles in the nucleotide sequence analyzed.

In another example, the method further comprises providing the set of primers and/or the analyzed primers and/or selected primers.

In another example, the method further comprises providing information pertaining to the sequences of the set of primers and/or the analyzed primers and/or selected primers e.g., in a computer-readable form or by way of an electronic medium or paper medium.

In one example, the first primer comprises a sequence having a Tm between about 60° C. and about 75° C., more preferably between about 60° C. and about 65° C., even more preferably about 61° C. or 62° C. or 63° C. or 64° C. or 65° C. It is preferred for first primers i.e., locus-specific primers to be capable of annealing to a nucleic acid template at a distance that is separated by about 400 bp, preferably about 450 bp or about 500 bp. Alternatively, or in addition, locus-specific piimer(s) comprise a 3′-nucleotide complementary to a SNP allele present in a nucleic acid template.

In one example, the Tm of the allele specific region of the second primer is between about 35° C. and about 50° C., more preferably between about 42° C. and about 48° C., even more preferably about 42° C. or 43° C. or 44° C. or 45° C. or 46° C. or 47° C. or 48° C.

Allele specific regions of the second primers are preferably between about 12 nucleotides and about 40 nucleotides in length, preferably between about 15 nucleotides in length and about 36 nucleotides in length, including about 18 nucleotides in length or about 21 nucleotides in length or about 24 nucleotides in length or about 27 nucleotides in length or about 30 nucleotides in length or about 33 nucleotides in length or about 36 nucleotides in length. It is also preferred for allele-specific regions of second primers to amplify a region in nucleic acid having a length between about 90 bp and about 300 bp in length, preferably between about 100 bp and about 250 bp in length or between about 100 bp and about 200 bp in length or between about 100 bp and about 150 bp in length.

The tag region is not complementary to a target DNA, and preferably comprises a sequence having a melting temperature of about 5° C. or 6° C. or 7° C. or 8° C. or 9° C. or 10° C. or 11° C.

The tag region is between about 2 nucleotides in length and about 9 nucleotides in length and has a melting temperature of about 5° C. or 6° C. or 7° C. or 8° C. or 9° C. or 10° C. or 11° C. Preferred forms of the tag region have a length between 2 nucleotides in length and about 8 nucleotides in length or between 2 nucleotides in length and about 7 nucleotides in length or between 2 nucleotides in length and about 6 nucleotides in length or between 2 nucleotides in length and about 5 nucleotides in length or between 2 nucleotides in length and about 4 nucleotides in length or 2 or 3 nucleotides in length.

The present invention also provides a computer-readable medium comprising information pertaining to the sequences of a panel of first primer(s) and/or second primer(s) that provide discrimination between alleles in nucleic acid comprising a sequence homologous to the nucleic acid template, wherein said information is obtained by a method of the invention described with reference to any embodiment or example hereof.

### General Information and Definitions

This specification contains nucleotide and amino acid sequence information prepared using PatentIn Version 3.4, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence are indicated by information provided in the numeric indicator fields <211>, <212> and <213>, respectively. Nucleotide sequences referred to in the specification are defined by the term “SEQ ID NO:”, followed by the sequence identifier (e.g. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and/or all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

### BRIEF DESCRIPTION OF THE DRAWINGS

FIG. 1 is a diagrammatic representation of a temperature switch PCR method of the present invention for allelic discrimination by differential amplification product size using a pair of allele specific (AS) primers designed to anneal to different DNA strands. Locus specific (LS) primers are labeled L₁ and L₂, and AS primers are labeled A1 and A2. AS primer A₁ is designed to anneal to the B allele of the polymorphism N. Expected PCR products for each genotype are also shown at the bottom of the figure.

FIG. 2 is a diagrammatic representation of a temperature switch PCR method of the present invention for allelic discrimination by differential product size using a pair of AS primers that anneal to the same DNA strand. LS primers are labeled L₁ and L₂, and AS primers are labeled A₁ and A₂. AS primer A₁ anneals to the A allele of polymorphism N, and AS primer A₂ anneals to the B allele of polymorphism B. AS primer A₁ has a longer tag region than AS primer A₂ thereby producing a longer PCR product than produced. Expected PCR products for each genotype are also shown at the bottom of the Figure.

FIG. 3a is a diagrammatic representation showing a temperature switch PCR method of the present invention for allelic discrimination by different product size using a single AS primer. LS primers are labeled L₁ and L₂, and the AS primer is labeled A₁. AS primer A₁ anneals to the B allele of polymorphism N. Expected PCR products for each genotype are also shown at the bottom of the Figure.

FIG. 3b is a diagrammatic representation showing a temperature switch PCR of the present invention for allelic discrimination using a single AS primer. LS primers are labeled LS₁ and LS₂ and AS primers are labeled AS₁ and AS₂. AS₁ anneals to allele A of polymorphism N and AS₂ anneals to allele B of polymorphism N. Two separate PCRs are performed, one with AS₁ to detect allele A and the other with AS₂ to detect allele B. Expected PCR products are also shown at the bottom of the figure.

FIG. 4 is a diagrammatic representation showing a temperature switch PCR of the present invention for allelic discrimination by differential product labeling using a pair of AS primers designed to the same DNA strand. LS primers are labeled L₁ and L₂, and AS primers are labeled A₁ and A₂. AS primer A₁ anneals to the A allele of polymorphism N, and AS primer A₂ anneals to the B allele of polymorphism B. AS primer A₁ comprises a detectable marker X that is different to the detectable marker Y linked to AS primer A₂. Expected PCR products and label(s) linked thereto for each genotype are also shown at the bottom of the Figure.

FIG. 5 is a graphical representation showing a temperature switch PCR of the present invention for allelic discrimination by differential product detection using a pair of AS primers designed to opposite DNA strands. LS primers are labeled L₁ and L₂, and AS primers are labeled A₁ and A₂. Allele specific primers amplify an amplification product and the allele at polymorphism N is detected using, for example, melting curve analysis. Expected PCR products are also shown at the bottom of the figure.

FIG. 6 is a diagrammatic representation showing a temperature switch PCR of the present invention for allelic discrimination by differential product detection using a single AS primer. LS primers are labeled L₁ and L₂, and the AS primer is labeled A₁. Amplification product from L₁ and A₁ are analyzed using, for example, melting curve analysis to detect the allele at polymorphism N. Expected PCR products are also shown at the bottom of the figure.

FIG. 7 is a copy of photographic representations showing PCR products amplified from eight barley lines using AS primers with the complementary region having a melting temperature of 40° C., 45° C. and 50° C., respectively (as indicated). The AS primers were designed for validated SNPs having allele A and allele B in a (a) putative gene located on chromosome 5H, and (b) nicotinate phosphoribosyltransferase-like gene. Solid arrows indicate the size of the expected PCR product.

FIG. 8 is a copy of a photographic representation showing amplicons produced using primer combinations set forth in Table 1 in reactions performed using temperature switch PCR of the present invention (TSP cycling) and standard PCR cycling conditions (standard cycling). PCR products are shown for reactions using primers designed for a putative gene located on chromosome 5H in barley. The barley lines tested (wells A-D) had the genotypes AA, BB, AB and AB, respectively. Numbers correspond to numbers in Table 1.

FIG. 9a is a graphical representation showing a temperature switch PCR of the present invention allelic discrimination by differential product size using a pair of AS primers designed to opposite DNA strands. The assays were performed in barley (Hordeum vulgare) using samples with the following zygosity in lanes 1-8 BB, BB, AA, AB, BB, AB, BB and BB. AS primers have a complementary region melting temperature of 40° C. Two reactions were performed for each sample, one with AS forward primers specific for allele A, and the other with AS forward primer specific for allele B. Primers were designed to assay SNPs in a putative gene located on chromosome 5H.

FIG. 9b is a copy of a photographic representation showing a temperature switch PCR of the present invention allelic discrimination by differential product size using a pair of AS primers designed to opposite DNA strands. The assays were performed in barley (Hordeum vulgare) using samples with the following zygosity in lanes 1-8 BB, AA, AA, AB, BB, AB, BB, AB. AS primers have a complementary region melting temperature of 40° C. Two reactions were performed for each sample, one with AS forward primers specific for allele A, and the other with AS forward primer specific for allele B. Primers were designed to assay SNPs in a nicotinate phosphoribosyltransferase-like gene.

FIG. 9b is a copy of a photographic representation showing a temperature switch PCR of the present invention allelic discrimination by differential product size using a pair of AS primers designed to opposite DNA strands. The assays were performed in barley (Hordeum vulgare) using samples with the following zygosity in lanes 1-8 AA, BB, BB, AB, AA, AB, AA, AB. AS primers have a complementary region melting temperature of 40° C. Two reactions were performed for each sample, one with AS forward primers specific for allele A, and the other with AS forward primer specific for allele B. Primers were designed to assay SNPs in a nicotinate phosphoribosyltransferase-like gene.

FIG. 10 is of a copy of a photographic representation showing results of a temperature switch PCR of the present invention configured for allelic discrimination by differential product size using a pair of AS primers designed to opposite DNA strands. The assay was performed using genomic DNA from bread wheat (Triticum aestivum) using samples with known zygosity. The AS forward and reverse primers A₁ and A₂ had complementary region melting temperatures of 50° C. and 40° C., respectively. Two reactions were performed for each sample, one using the AS forward primer specific for allele A, and the other using AS forward primer specific for allele B. Primers were designed to assay a SNP located in a putative nodulin gene on the chromosome 3B.

FIG. 11 is a copy of a photographic representation showing results of a temperature switch PCR of the present invention configured for allelic discrimination by differential product detection using a pair of AS primers designed to opposite DNA strands. The assay was performed in barley (Hordeum vulgare) using samples with known zygosity. The AS primers have a complementary region melting temperature of 40° C. Primers were designed to assay a SNP in a nicotinate phosphoribosyltransferase-like gene.

FIG. 12 provides graphical representations showing biphasic accumulation of reaction product in TSP assays performed using real-time PCR during the final 45 cycles of amplification. TSP assays were performed in barley (Hordeum vulgare) using samples with known zygosity. Allele specific (AS) primers have a complementary region melting temperature of 45° C., a 3′-nucleotide complementary to the SNP allele present at the locus, and a non-complementary 5′-tag designed to increase the melting temperature of the AS primer to 53° C. once the non-complementary sequence is incorporated into PCR product. Primers were designed to assay SNPs in genes encoding (a) putative Rieske Fe—S precursor protein, (b) fructose-6-phosphate 2-kinase, (c), unnamed protein product from rice, and (d) cytosolic aldehyde dehydrogenase. Numbers for each curve in each panel represent different reactions obtained using the following primer combinations: 1 is primer Combination L1 and L2; 2 is primer combination L1 L2 A1 and A2; 3 is primer combination A1 and A2; and 4 is no primer, wherein L1 is an LS forward primer; L2 is LS reverse primer; A1 is AS forward primer specific for allele A and A2 is AS reverse primer. Data demonstrate efficient transition from the amplification of LS product to the accumulation of AS product in the second phase of the reaction and efficient annealing of AS primers to the enriched target sequence (LS product) at the second phase annealing temperature, allowing for highly efficient self-amplification of AS product in subsequent cycles due to incorporation of the non-complementary 5′-tail, and therefore, out-competing of the accumulation of LS product.

FIG. 13 is a copy of a photographic representation showing results of TSP amplificatin of methylenetetrahydrofolate reductase alleles comprising 677C or 677T resolved using 2% (w/v) agarose gel electrophoresis and stained using ehtidium bromide. Lanes from left to right are as follows: Lane 1, a 1.1 kb plus ladder (Invitrogen; 100 bp increment bands); lanes 2-13 show alleles in the MTHFR gene, wherein lanes 2, 7, 9, 11 and 12 show two copies of the 677C allele, lanes 4, 6, 8 show two copies of the 677T allele, and lanes 3, 5, 10 and 13 show one copy of the 677C allele and one copy of the 677T allele in the sample DNA.

### DETAILED DESCRIPTION OF THE PREFERRED EMBODIMENTS

### Primer Design

In one example of the present invention, a first primer or an allele-specific region of a second primer is designed such that it comprises a sequence having at least about 80% identity overall to a strand of a template nucleic acid. More preferably, the degree of sequence identity is at least about 85% or 90% or 95% or 98% or 99%. For example, the primer or a region of a primer may comprise a sequence having at least about 80% identity to a strand of a locus of interest.

Clearly, the specific composition of a primer of the invention (or more specifically, a first primer or an allele-specific region of a second primer) will depend upon the sequence of the template nucleic acid of interest. Accordingly, the sequence of a first primer or an allele-specific region of a second primer is not to be taken to be limited to a particular sequence. Rather the sequence need only be sufficient to allow for annealing of the first primer or allele-specific region of a second primer to a template nucleic acid and initiation of an amplification reaction.

For a first primer of the invention, as a primer is generally extended in the 5′- to 3′-direction it is preferred that at least the 3′-terminal nucleotide is complementary to the relevant nucleotide in the template nucleic acid. More preferably, at least the 3 or 4 or 6 or 8 or 10 contiguous nucleotides at the 3′-terminus of the primer are complementary to the relevant nucleotides in the template nucleic acid. The complementarity of the 3′ terminus of the primer ensures that the extending end of the primer is capable of initiating amplification of the template nucleic acid, for example, by a polymerase.

As for an allele-specific primer, in some methods described herein a 3′ nucleotide of said region is complementary to an allele of a polymorphism or a mutation. Accordingly, the 3′ nucleotide will also be non-complementary to another allele of the polymorphism or mutation. Such a primer is useful for only amplifying nucleic acid to a detectable level in the presence of the allele complementary to the 3′ nucleotide of the allele-specific region.

In some embodiments of the present invention, an allele specific region additionally comprises a nucleotide that is non-complementary to a template nucleic acid or first amplification product, said non-complementary nucleotide being positioned at nucleotide position −2 or −3 from the 3′ terminus of the allele specific region. Suitable non-complementary nucleotides will be apparent to the skilled artisan and/or are described, for example, in Little et al., In: Taylor (ed) Laboratory Methods for the Detection of Mutations and Polymorphisms in DNA, CRC Press, Boca Raton, Fla., USA, pp. 45-51. For example, in the case of a strong ‘mismatch’ (non-complementary nucleotide) (G/A or C/T mismatch) at the 3′ terminus of an allele specific region the additional non-complementary nucleotide can be a ‘weak’ mismatch (C/A or G/T), and vice versa. In the presence of a ‘medium’ mismatch (A/A, C/C/ G/G or T/T) at the 3′ terminal nucleotide of the allele-specific region, the additional non-complementary nucleotide can also be a ‘medium’ mismatch.

As regions of non-complementarity reduce the predicted Tm of a primer and may be associated with amplification of non-template nucleic acid it is preferred that a primer of the invention does not comprise multiple contiguous nucleotides that are not identical to a strand of the template nucleic acid. Preferably, the primer comprises no more than 6 or 5 or 4 or 3 or 2 contiguous nucleotides that are not identical to a strand of the template nucleic acid. More preferably, any nucleotides that are not identical to a strand of the template nucleic acid are non-contiguous.

To determine whether or not two nucleotide sequences fall within a particular percentage identity limitation recited herein, those skilled in the art will be aware that it is necessary to conduct a side-by-side comparison or multiple alignment of sequences. In such comparisons or alignments, differences may arise in the positioning of non-identical residues, depending upon the algorithm used to perform the alignment. In the present context, reference to a percentage identity between two or more nucleotide sequences shall be taken to refer to the number of identical residues between said sequences as determined using any standard algorithm known to those skilled in the art. For example, nucleotide sequences may be aligned and their identity calculated using the BESTFIT program or other appropriate program of the Computer Genetics Group, Inc., University Research Park, Madison, Wis., United States of America (Devereaux et al, Nucl. Acids Res. 12, 387-395, 1984).

Alternatively, a suite of commonly used and freely available sequence comparison algorithms is provided by the National Center for Biotechnology Information (NCBI) Basic Local Alignment Search Tool (BLAST) (Altschul et al. J. Mol. Biol. 215: 403-410, 1990), which is available from several sources, including the NCBI, Bethesda, Md. The BLAST software suite includes various sequence analysis programs including “blastn,” that is used to align a known nucleotide sequence with other polynucleotide sequences from a variety of databases. Also available is a tool called “BLAST 2 Sequences” that is used for direct pairwise comparison of two nucleotide sequences.

As used herein the term “NCBI” shall be taken to mean the database of the National Center for Biotechnology Information at the National Library of Medicine at the National Institutes of Health of the Government of the United States of America, Bethesda, Md., 20894.

Generally, a primer comprises or consists of at least about 10 nucleotides, more preferably at least about 12 nucleotides or at least about 15 or 20 nucleotides that anneal to a nucleic acid template or are complementary to the nucleic acid template. However, longer primers are also used in PCR reactions, for example, reactions in which a long region of nucleic acid (e.g., greater than 1000 bp) is amplified. Accordingly, the present invention additionally contemplates a primer comprising at least about 25 or 30 or 35 nucleotides that anneal to a nucleic acid template or are complementary to the nucleic acid template.

Alternatively, a primer comprising one or modified bases, such as, for example, locked nucleic acid (LNA) or peptide nucleic acid (PNA) need only comprise a region of at least about 8 nucleotides that anneal to a nucleic acid template or are complementary to the nucleic acid template. Preferably, the complementary nucleotides are contiguous.

As will be apparent to the skilled artisan, the number of nucleotides capable of annealing to a nucleic acid template is related to the stringency under which the primer will anneal. Preferably, a primer of the invention anneals to a nucleic acid template under moderate to high stringency conditions.

In one embodiment, the stringency under which a primer of the invention anneals to a template nucleic acid is determined empirically. Generally, such a method requires performance of an amplification reaction using one or more primers under various conditions and determining the level of specific amplification produced.

Alternatively, a primer of the invention is labeled with a detectable marker (e.g., a radionucleotide or a fluorescent marker) and the level of primer that has annealed to a target nucleic acid under suitably stringent conditions is determined.

For the purposes of defining the level of stringency, a moderate stringency annealing conditions will generally be achieved using a condition selected from the group consisting of:
(i) an incubation temperature between about 42° C. and about 55° C.;
(ii) an incubation temperature between about 15° C. and 10° C. less than the predicted Tm for a primer; and
(iii) a Mg²⁺ concentration of between about 2 mM and 3 mM.

High stringency annealing conditions will generally be achieved using a condition selected from the group consisting of:

(i) an incubation temperature above about 55° C. and preferably above about 65° C.;
(ii) an incubation temperature between about 10° C. and 1° C. less than the predicted Tm for a primer; and
(iii) a Mg²⁺ concentration of between about 1 mM and 1.9 mM.

Alternative or additional conditions for enhancing stringency of annealing will be apparent to the skilled artisan. For example, a reagent such as, for example, glycerol (5-10%), DMSO (2-10%), formamide (1-5%), Betaine (0.5-2M) or tetramethylammonium chloride (TMAC, >50 mM) are known to alter the annealing temperature of a primer and a nucleic acid template (Sarkar et al., Nucl. Acids Res. 18: 7465; 1990, Baskaran et al. Genome Res. 6: 633-638, 1996; and Frackman et al., Promega Notes 65: 27, 1998).

Conditions for altering the stringency of a PCR reaction are understood by those skilled in the art. For the purposes of further clarification only, reference to the parameters affecting annealing between nucleic acid molecules is found in Ausubel et al. (Current Protocols in Molecular Biology, Wiley Interscience, ISBN 047150338, 1992), which is herein incorporated by reference.

Alternatively, the conditions under which a primer anneals to a nucleic acid template are determined in silico. For example, methods for determining the predicted melting temperature (or Tm) of a primer (or the temperature at which a primer denatures from a specific nucleic acid) are known in the art.

For example, the method of Wallace et al., (Nucleic Acids Res. 6, 3543, 1979) estimates the Tm of a primer based on the G, C, T and A content. In particular, the described method uses the formula 2(A+G)+4(G+C) to estimate the Tm of a probe or primer.

Alternatively, the nearest neighbor method described by Breslauer et al., Proc. Natl. Acad. Sci. USA, 83:3746-3750, 1986 is useful for determining the Tm of a primer. The nearest neighbour method uses the formula:

Tₘ(calc)=ΣΔH⁰/(Rln(Cₜ/n)+ΣΔS⁰),

wherein ΔH⁰ is standard enthalpy for helix formation, ΔS⁰ is standard entropy for helix formation, Cₜ is the total strand concentration, n reflects the symmetry factor, which is 1 in the case of self-complementary strands and 4 in the case of non-self-complementary strands and R is the gas constant (1.987).

Ryuchlik et al., Nucl. Acids Res. 18: 6409-6412, 1990 described an alternative formula for determining Tm of an oligonucleotide:

$${Tm}^{primer} = \frac{d H}{d S + R ln ( c / 4 )} + 16.61 g \frac{[ {K}^{+} ]}{1 + 0.7 [ {K}^{+} ]} - 273.15 ,$$

wherein, dH is enthalpy for helix formation, dS is entropy for helix formation, R is molar gas constant (1.987 cal/° C. mol), “c” is the nucleic acid molar concentration (determined empirically, W. Rychlik et. al., supra), (default value is 0.2 μM for unified thermodynamic parameters), [K⁺] is salt molar concentration (default value is 50 mM).

Suitable software for determining the Tm of an oligonucleotide using the nearest neighbor method is known in the art and available from, for example, US Department of Commerce, Northwest Fisheries Service Center and Department of Molecular Genetics and Biochemistry, University of Pittsburgh School of Medicine.

Alternatively, for longer primers (i.e., a primer comprising at least about 200 nucleotides), the method of Meinkoth and Wahl (In: Anal Biochem, 138: 267-284, 1984), is useful for determining the Tm of the primer. This method uses the formula:

81.5+16.6(log₁₀M)+0.41(% GC)−0.61(% form)−500/Length in bp,

wherein M is the molarity of Na+ and % form is the percentage of formamide (set to 50%)

For a primer that comprises or consists of PNA the Tm is determined using the formula (described by Giesen et al., Nucl. Acids Res., 26: 5004-5006):

T_{mpred}=c₀+c₁*T_{mnnDNA}+c₂*f_{pyr}+c₃*length,

wherein, in which T_{mnnDNA} is the melting temperature as calculated using a nearest neighbor model for the corresponding DNA/DNA duplex applying ΔH⁰ and ΔS⁰ values as described by SantaLucia et al. Biochemistry, 35: 3555-3562, 1995. f_{pyr} denotes the fractional pyrimidine content, and length is the PNA sequence length in bases. The constants are c₀=20.79, c₁=0.83, c₂=−26.13 and c₃=0.44

To determine the Tm of a primer comprising one or more LNA residues a modified form of the formula of SantaLucia et al. Biochemistry, 35: 3555-3562, 1995 is used:

$$Tm = \frac{Δ H}{Δ S + ln ( {[ Na ]}^{0.36} * {( C / 4 )}^{1 , 987} )} ,$$

A suitable program for determining the Tm of a primer comprising LNA is available from, for example, Exiqon, Vedbaek, Germany.

A temperature that is similar to (e.g., within 5° C. or within 10° C.) or equal to the proposed/estimated temperature at which a primer denatures from a template nucleic acid is considered to be high stringency. Medium stringency is to be considered to be within 10° C. to 20° C. or 10° C. to 15° C. of the calculated Tm of the probe or primer.

A primers or primer sequence that is predicted to be or shown to be capable of selectively annealing to a nucleic acid template is also optionally analyzed for one or more additional characteristics that make it suitable for use as a primer in the method of the invention. For example, a primer is analyzed to ensure that it is unlikely to form secondary structures (i.e., the primer does not comprise regions of self-complementarity).

Furthermore, should the primer be proposed to be used in a reaction with one or more other primers (e.g., a PCR reaction and/or a multiplex reaction) all primers may be assessed to determine their ability to anneal to one another and form “primer dimers”. Methods for determining a primer that is capable of self-dimerization and/or primer dimer formation are known in the art and/or described supra.

Methods for designing and/or selecting a primer suitable for use in an amplification reaction are known in the art and described, for example, in Innis and Gelfand (1990) (In: Optimization of PCRs. pp. 3-12 in: PCR Protocols (Innis, Gelfand, Sninsky and White, eds.); Academic Press, New York) and Dieffenbach and Dveksler (Eds) (In: PCR Primer: A Laboratory Manual, Cold Spring Harbor Laboratories, NY, 1995). Such methods are particularly suited, for example, for designing a locus specific sequence of a primer of the invention.

Generally, it is recommended that a primer satisfies the following criteria:
(i). the primer comprises a region that is to anneal to a target sequence having at least about 17-28 bases in length;
(ii). the primer comprises about 50-60% (G+C);
(iii) the 3′-terminus of the primer is a G or C, or CG or GC (this prevents “breathing” of ends and increases efficiency of initiation of amplification);
(iv) preferably, the primer has a Tm between about 55 and about 80° C.;
(v) the primer does not comprise three or more contiguous Cs and/or Gs at the 3′-ends of primers (as this may promote mispriming at G or C-rich sequences due to the stability of annealing);
(vi) the 3′-end of a primer should not be complementary with another primer in a reaction; and
(vii) the primer does not comprise a region of self-complementarity.

Several software programs are available that enable the design of one or more primers, or a region of a primer (e.g., a locus specific sequence of a first primer of the invention). For example, a program selected from the group consisting of:
(i) Primer3, available from the Center for Genome Research, Cambridge, Mass., USA, designs one or more primers for use in an amplification reaction based upon a known template sequence;
(ii) Primer Premier 5, available from Biosoft International, Palo Alto, Calif., USA, designs and/or analyzes primers;
(iii) CODEHOP, available from Fred Hutchinson Cancer Research Centre, Seattle, Wash., USA, designs primers based on multiple protein alignments; and
(iv) FastPCR, available from Institute of Biotechnology, University of Helsinki, Finland, designs multiple primers, including primers for use in a multiplex reaction, based on one or more known sequences.

When designing a primer of the invention, the composition of the template nucleic acid is considered (i.e. the nucleotide sequence) as is the type of amplification reaction to be used. For example, should allele specific PCR be used, the 3′ nucleotide of one of the primers used in such a reaction corresponds to the site of an allele of interest, such as, for example a SNP. In this manner only in the presence of a nucleotide that is complementary to that in the primer does annealing occur and amplification achieved.

Furthermore, should the primer be used in a multiplex reaction it is preferred that the amplification product produced is not similar in molecular weight to that produced using another primer or set thereof thereby rendering detection difficult. Accordingly, it is preferred that there is sufficient difference in molecular weight in amplified products to enable detection using a technique known in the art, such as, for example, gel electrophoresis or mass spectrometry.

While it is preferable to produce amplification products of distinct molecular weights, by using differential labeling with different detectable markers, products of similar length are resolved. Accordingly, it is not essential that each of the nucleic acids amplified using the method of the invention is different molecular weight.

### Tag Regions

The tag region in a second primer of the invention serves the dual purpose of enhancing the specificity annealing of the second primer and increasing the temperature at which a second primer anneals to a nucleic acid following incorporation of the tag region into a nucleic acid.

The length and/or nucleotide composition of the tag region depends, in part, on the temperature at which an allele specific region anneals to a nucleic acid and the temperature at which a first primer anneals to a nucleic acid and/or the temperature at which a PCR is performed to produce a third amplification product.

A tag region that is unable to anneal to the template nucleic acid is selected to ensure that it does not cause non-specific annealing of the first primer in the first amplification reaction and the amplification of non-template nucleic acid. Preferably, the tag region is unable to anneal to a nucleic acid in a sample being assayed to such a degree as to amplify nucleic acid to a detectable level (i.e. background amplification).

As will be apparent to the skilled artisan, the requirement that the tag region not anneal to a template nucleic acid does not require that the tag region not anneal under any conditions. Rather, it is preferred that the tag region is not capable of annealing to the template nucleic acid under conditions sufficient for annealing of the locus specific sequence to the template nucleic acid. For example, the tag region may anneal to the template nucleic acid under low stringency conditions.

In one embodiment, it is preferred that the tag comprises a sequence of nucleotides that does not naturally occur in a sample being assayed. Methods for determining a sequence that is not present in a sample being assayed will be apparent to the skilled artisan. For example, the nucleotide sequence of the tag region is analyzed using a program, such as, for example, BLAST to determine whether or not that sequence (or its complement) occurs naturally in an organism being assayed.

Preferred tags comprise a high G+C content. Such a high G+C content means that a short tag region is required to sufficiently increase the Tm of a second primer. For example, a tag region comprises at least about 70% G and/or C, or at least about 80% G and/or C, or at least about 90% G and/or C, or at least about 100% G and/or C.

Examples of sequences of suitable tag regions include, for example:

GG
GC
CG
CC
GCG
CGC
GGC
CCG
GCC
GCGG
GGCG
GCGC
GGGC
GCCG
GGCC
GCGG
CGCG
CCGC
CGGC
CCCG
CGCC
CCGG
GCCCGCG
GGCGGCGG
CCCGCG
GGCGC
GCGCCG
GCCCG
CCGCCC
CCCG
GGCCG
GGGGCGGGG

Preferred tags are 2 to about 9 nucleotides in length or from 2 to about 8 nucleotides in length or from 2 to about 7 nucleotides in length or from 2 to about 6 nucleotides in length or from 2 to about 5 nucleotides in length or from 2 to about 4 nucleotides in length or 2 or 3 nucleotides in length.

However, the present invention is not to be limited to a tag region comprising any specific sequence.

### Primer Synthesis

Following primer design and or analysis, a specific the primer is produced and/or synthesized. Methods for producing/synthesizing a primer are known in the art. For example, oligonucleotide synthesis is described, in Gait (Ed) (In: Oligonucleotide Synthesis: A Practical Approach, IRL Press, Oxford, 1984). For example, a probe or primer may be obtained by biological synthesis (e.g. by digestion of a nucleic acid with a restriction endonuclease) or by chemical synthesis. For short sequences (up to about 100 nucleotides) chemical synthesis is preferable.

In one embodiment, a primer comprising deoxynucleotides (e.g., a DNA based oligonucleotide) is produced using standard solid-phase phosphoramidite chemistry. Essentially, this method uses protected nucleoside phosphoramidites to produce an oligonucleotide of up to about 80 nucleotides. Typically, an initial 5′-protected nucleoside is attached to a polymer resin by its 3′-hydroxy group. The 5′ hydroxyl group is then de-protected and the subsequent nucleoside-3′-phosphoramidite in the sequence is coupled to the de-protected group. An internucleotide bond is then formed by oxidizing the linked nucleosides to form a phosphotriester. By repeating the steps of de-protection, coupling and oxidation an oligonucleotide of desired length and sequence is obtained. Suitable methods of oligonucleotide synthesis are described, for example, in Caruthers, M. H., et al., “Methods in Enzymology,” Vol. 154, pp. 287-314 (1988).

Other methods for oligonucleotide synthesis include, for example, phosphotriester and phosphodiester methods (Narang, et al. Meth. Enzymol 68: 90, 1979) and synthesis on a support (Beaucage, et al Tetrahedron Letters 22: 1859-1862, 1981), and others described in “Synthesis and Applications of DNA and RNA,” S. A. Narang, editor, Academic Press, New York, 1987, and the references contained therein.

For longer sequences standard replication methods employed in molecular biology are useful, such as, for example, the use of M13 for single stranded DNA as described by J. Messing (1983) Methods Enzymol, 101, 20-78.

Alternatively, a plurality of primers are produced using standard techniques, each primer comprising a portion of a desired primer and a region that allows for annealing to another primer. The primers are then used in an overlap extension method that comprises allowing the primers to anneal and synthesizing copies of a complete primer using a polymerase. Such a method is described, for example, by Stemmer et al., Gene 164, 49-53, 1995.

As discussed supra a primer of the invention may also include one or more nucleic acid analogs. For example, a primer comprises a phosphate ester analog and/or a pentose sugar analog. Alternatively, or in addition, a primer of the invention comprises polynucleotide in which the phosphate ester and/or sugar phosphate ester linkages are replaced with other types of linkages, such as N-(2-aminoethyl)-glycine amides and other amides (see, e.g., Nielsen et al., Science 254: 1497-1500, 1991; WO 92/20702; and U.S. Pat. No. 5,719,262); morpholinos (see, for example, U.S. Pat. No. 5,698,685); carbamates (for example, as described in Stirchak & Summerton, J. Org. Chem. 52: 4202, 1987); methylene(methylimino) (as described, for example, in Vasseur et al., J. Am. Chem. Soc. 114: 4006, 1992); 3′-thioformacetals (see, for example, Jones et al., J. Org. Chem. 58: 2983, 1993); sulfamates (as described, for example in, U.S. Pat. No. 5,470,967); 2-aminoethylglycine, commonly referred to as PNA (see, for example, WO 92/20702). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate. Methods for the production of a primer comprising such a modified nucleotide or nucleotide linkage are known in the art and discussed in the documents referred to supra.

For example, a primer of the invention comprises one or more LNA and/or PNA residues. Primers comprising one or more LNA or PNA residues have been previously shown to anneal to nucleic acid template at a higher temperature than a primer that comprises substantially the same sequence but does not comprise the LNA or PNA residues.

Methods for the synthesis of an oligonucleotide comprising LNA are described, for example, in Nielsen et al, J. Chem. Soc. Perkin Trans., 1: 3423, 1997; Singh and Wengel, Chem. Commun. 1247, 1998. Methods for the synthesis of an oligonucleotide comprising are described, for example, in Egholm et al., Am. Chem. Soc., 114: 1895, 1992; Egholm et al., Nature, 365: 566, 1993; and Orum et al., Nucl. Acids Res., 21: 5332, 1993.

As described herein, a second primer can additionally comprise a detectable marker (for example, a fluorescent dye) to enable detection of an amplification product produced using the method of the invention. Accordingly, in one embodiment, at least one primer of the invention comprises or is conjugated to a detectable marker. As used herein, the term “detectable marker” refers to any moiety which can be attached to a primer of the invention and: (i) provides a detectable signal; (ii) interacts with a second detectable marker to modify the detectable signal provided by the second detectable marker, e.g. FRET (Fluorescent Resonance Energy Transfer); (iii) stabilize annealing, e.g., duplex formation; or (iv) provide a member of a binding complex or affinity set, e.g., affinity, antibody/antigen, ionic complexation, hapten/ligand, e.g. biotin/avidin.

Labeling of a primer is accomplished using any one of a large number of known techniques employing known detectable markers, linkages, linking groups, reagents, reaction conditions, and analysis and purification methods. Detectable markers include, but are not limited to, light-emitting or light-absorbing compounds which generate or quench a detectable fluorescent, chemiluminescent, or bioluminescent signal (for example, as described in Kricka, L. in Nonisotopic DNA Probe Techniques (1992), Academic Press, San Diego, pp. 3-28). Fluorescent reporter dyes useful for labeling biomolecules include, but are not limited to, fluoresceins (see, for example U.S. Pat. Nos. 5,188,934; 6,008,379; or U.S. Pat. No. 6,020,481), rhodamines (as described, for example, in U.S. Pat. No. 5,366,860; U.S. Pat. No. 5,847,162; U.S. Pat. No. 5,936,087; or U.S. Pat. No. 6,051,719), benzophenoxazines (for example, as described in U.S. Pat. No. 6,140,500), energy-transfer fluorescent dyes, comprising pairs of donors and acceptors (as described in U.S. Pat. No. 5,863,727; U.S. Pat. No. 5,800,996; or U.S. Pat. No. 5,945,526), or a cyanine (as described, for example, in WO 97/45539). Exemplary fluorescein dyes include, but are not limited to, 6-carboxyfluorescein; 2′,4′,1,4-tetrachlorofluorescein; and 2′,4′,5′,7′,1,4-hexachlorofluorescein. Detectable markers also include, but are not limited to, semiconductor nanocrystals, or Quantum Dots® (as described, for example in U.S. Pat. No. 5,990,479 or U.S. Pat. No. 6,207,392). Suitable methods for linking a detectable marker to a primer (or labeling a primer) are also described in the references supra.

Alternatively, or in addition, a primer is produced with a fluorescent nucleotide analog to facilitate detection. For example, coupling allylamine-dUTP to the succinimidyl-ester derivatives of a fluorescent dye or a hapten (such as biotin or digoxigenin) enables preparation of many common fluorescent nucleotides. Such a method is described in, for example Henegariu, Nat. Biotechnol. 18:345-348, 2000. Other fluorescent nucleotide analogs are also known in the art and described, for example, Jameson, Methods Enzymol. 278:363-390, 1997 or U.S. Pat. No. 6,268,132. Such nucleotide analogs are incorporated into nucleic acids, e.g., DNA and/or RNA, or oligonucleotides, via either enzymatic or chemical synthesis (e.g., a method described supra).

In one preferred example of the present invention, a primer is labeled with a fluorescent dye, such as, for example, 6-carboxyfluorescein (FAM), VIC, NED or PET. To label a primer with a fluorescent dye a simple two-step process is used. In the first step, an amine-modified nucleotide, 5-(3-aminoallyl)-dUTP, is incorporated into DNA using conventional enzymatic labeling methods. This step ensures relatively uniform labeling of the probe with primary amine groups. In the second step, the amine-modified DNA is chemically labeled using an amine-reactive fluorescent dye. Various commercial kits for labeling a primer are known in the art and available from, for example, Molecular Probes (Invitrogen detection Technology) (Eugene, Oreg., USA) or Applied Biosystems (Foster City, Calif., USA).

Commercial sources for the production of a labeled probe or primer or for a suitable label will be known to the skilled artisan, e.g., Sigma-Genosys, Sydney, Australia or Applied Biosystems, Foster City, Calif., USA.

Using any method for oligonucleotide synthesis described herein and/or known in the art a set of first primers and/or a second primer or set thereof is synthesized.

In another example, a second primer is produced by coupling an oligonucleotide comprising a tag region to an oligonucleotide comprising an allele-specific region. For example, an oligonucleotide comprising a tag region is linked to another oligonucleotide using a RNA ligase, such as, for example T4 RNA ligase (as available from New England Biolabs). An RNA ligase catalyzes ligation of a 5′ phosphoryl-terminated nucleic acid donor to a 3′ hydroxyl-terminated nucleic acid acceptor through the formation of a 3′-5′ phosphodiester bond, with hydrolysis of ATP to AMP and PPᵢ. Suitable methods for the ligation of DNA and/or RNA molecules using a RNA ligase are known in the art and/or described in Ausubel et al (In: Current Protocols in Molecular Biology. Wiley Interscience, ISBN 047 150338, 1987) and Sambrook et al (In: Molecular Cloning: Molecular Cloning: A Laboratory Manual, Cold Spring Harbor Laboratories, New York, Third Edition 2001).

The present invention additionally provides a first and/or second primer of the invention, for example, as produced using a method known in the art and/or described herein.

Clearly the present invention additionally contemplates a kit comprising one or more first primers and/or one or more second primers. The kit optionally comprises reagents suitable for amplification of a nucleic acid using the method of the invention (e.g., a buffer and/or one or more deoxynucleotides and/or a polymerase). Optionally, the kit is packaged with instructions for use.

### Nucleic Acid Amplification

The method of the present invention is based on the amplification of a template nucleic acid using multiple rounds of PCR in a single reaction vessel. Accordingly, this single reaction vessel contains all of the components required for the performance of the multiple PCRs. Reagents required for a PCR are known in the art and include for example, one or more primers (described herein), a suitable polymerase, deoxynucleotides and/or ribonucleotides, a buffer. Suitable reagents are described for example, in Dieffenbach (ed) and Dveksler (ed) (In: PCR Primer: A Laboratory Manual, Cold Spring Harbor Laboratories, NY, 1995).

For example, a suitable polymerase for use in the method of the invention include, a DNA polymerase, a RNA polymerase, a reverse transcriptase, a T7 polymerase, a SP6 polymerase, a T3 polymerase, Sequenase™, a Klenow fragment, a Taq polymerase, a Taq polymerase derivative, a Taq polymerase variant, a Pfu polymerase, a Pfx polymerase, a Tfi polymerase, an AmpliTaq™ FS polymerase, a thermostable DNA polymerase with minimal or no 3′-5′ exonuclease activity, or an enzymatically active variant or fragment of any of the above polymerases. Preferably, a polymerase used in the method of the invention is a thermostable polymerase.

In one example, a mixture of two or more polymerases is used. For example, the mixture of a Pfx or Pfu polymerase and a Taq polymerase has been previously shown to be useful for amplifying templates comprising a high GC content or for amplifying a large template.

Suitable commercial sources for a polymerase useful for the performance of the invention will be apparent to the skilled artisan and include, for example, Stratagene (La Jolla, Calif., USA), Promega (Madison, Wis., USA), Invitrogen (Carlsbad, Calif., USA), Applied Biosystems (Foster City, Calif., USA) and New England Biolabs (Beverly, Mass., USA).

Methods of PCR are known in the art and described, for example, in Dieffenbach (ed) and Dveksler (ed) (In: PCR Primer: A Laboratory Manual, Cold Spring Harbor Laboratories, NY, 1995). Generally, for PCR two non-complementary nucleic acid primers comprising at least about 8, more preferably, at least about 15 or 20 nucleotides are annealed to different strands of a template nucleic acid, and amplicons of the template are amplified enzymatically using a polymerase, preferably, a thermostable DNA polymerase.

The present invention additionally contemplates RT-PCR. For RT-PCR, RNA is reverse transcribed using a reverse transcriptase (such as, for example, Moloney Murine Leukemia Virus) to produce cDNA. In this regard, the reverse transcription of the RNA is primed using, for example, a random primer (e.g., a hexa-nucleotide random primer) or oligo-dT (that binds to a poly-adenylation signal in mRNA). Alternatively, a locus-specific primer is used to prime the reverse transcription (e.g., a first primer of the invention). A sample is heated to ensure production of single stranded nucleic acid and then cooled to enable annealing of the primer. The sample is then incubated under conditions sufficient for reverse-transcription of the nucleic acid adjacent to an annealed primer by a reverse transcriptase. Following reverse transcription, the cDNA is used as a template nucleic acid for a PCR reaction, e.g., as described supra.

### Detecting Amplified Nucleic Acid

In one example, an amplification product, e.g., a third amplification product produced using the method of the present invention is/are separated using gel electrophoresis. The separated amplification product(s) is(are) then detected using a detectable marker that selectively binds nucleic acid, such as, for example, ethidium bromide, 4′-6-diamidino-2-phenylinodole (DAPI), methylene blue or SYBR® green I or II (available from Sigma Aldrich). Suitable methods for detection of a nucleic acid using gel electrophoresis are known in the art and described, for example, in Ausubel et al (In: Current Protocols in Molecular Biology. Wiley Interscience, ISBN 047 150338, 1987) and Sambrook et al (In: Molecular Cloning: Molecular Cloning: A Laboratory Manual, Cold Spring Harbor Laboratories, New York, Third Edition 2001).

In one example, the nucleic acid is separated using one dimensional agarose, agarose-acrylamide or polyacrylamide gel electrophoresis. Such separation techniques separate nucleic acids on the basis of molecular weight.

Alternatively, an amplification product is separated using two dimensional electrophoresis and detected using a detectable marker (e.g., as described supra). Two dimensional agarose gel electrophoresis is adapted from the procedure by Bell and Byers Anal. Biochem. 130:527, 1983. The first dimension gel is run at low voltage in low percentage agarose to separate DNA molecules in proportion to their mass. The second dimension is run at high voltage in a gel of higher agarose concentration in the presence of ethidium bromide so that the mobility of a non-linear molecule is drastically influenced by its shape.

Alternatively, or in addition, an amplification product is characterized or isolated using capillary electrophoresis. Capillary electrophoresis is reviewed in, for example, Heller, Electrophoresis 22:629-43, 2001; Dovichi et al., Methods Mol Biol 167:225-39, 2001; Mitchelson, Methods Mol Biol 162:3-26, 2001; or Dolnik, J Biochem Biophys Methods 41:103-19, 1999. Capillary electrophoresis uses high voltage to separate molecules according to their size and charge. A voltage gradient is produced in a column (i.e. a capillary) and this gradient drives molecules of different sizes and charges through the tube at different rates.

In another example, an amplification product is detected using an automated system, such as, for example, as produced by eGene, Inc. An example of such a system is described, for example, in Szantai et al., Clinical Chemistry 52: 1756-1762, 2006.

Alternatively, or in addition, one or more amplification products is detected using a microplate array diagonal gel electrophoresis (MADGE) method, e.g., as described in U.S. Pat. No. 6,071,396.

Alternatively, an amplification product is identified and/or isolated using chromatography. For example, ion pair-reversed phase HPLC has been shown to be useful for isolating a PCR product (Shaw-Bruha and Lamb, Biotechniques. 28:794-7, 2000.

Rather than contacting amplified nucleic acid with a detectable marker, the present invention additionally contemplates using a primer that comprises a detectable marker to facilitate detection of an amplification product. For example, a second primer of the invention is labeled with a detectable marker using a method known in the art and/or described herein and used in the method of the invention.

Amplified nucleic acid is then readily detected by detecting the label. In the case of a radiolabeled primer, the detection technique may comprise, for example, the use of a photographic film. In the case of a fluorescently labeled primer, the nucleic acid is detected, for example, by exposing a gel on which an amplification product has been resolved to light of a suitable wavelength to excite the label and detecting the fluorescence produced therefrom.

In another embodiment, the amplified nucleic acid is detected using, for example, mass spectrometry (e.g., MALDI-TOF). For example, a sample comprising nucleic acid amplified using the method of the invention is incorporated into a matrix, such as for example 3-hydroxypropionic acid, α-cyano-4-hydroxycinnamic acid, 3,5 dimethoxy-4-hydroxycinnamic acid (Sinapinic acid) or 2,5 dihydroxybenzoic acid (Gentisic acid). The sample and matrix are then spotted onto a metal plate and subjected to irradiation by a laser, promoting the formation of molecular ions. The mass of the produced molecular ion is analyzed by its time of flight (TOF), essentially as described by Yates, J. Mass Spectrom. 33, 1-19, 1998 and references cited therein. A time of flight instrument measures the mass to charge ratio (m/z) ratio of an ion by determining the time required for it to traverse the length of a flight tube. Optionally, such a TOF mass analyzer includes an ion mirror at one end of the flight tube that reflects said ion back through the flight tube to a detector. Accordingly, an ion mirror serves to increase the length of a flight tube, increasing the accuracy of this form of analysis. By determining the time of flight of the ion, the molecular weight of an amplified nucleic acid is determined.

The advantage of this form of technique is that an amplification product is detected and characterized without the requirement for labeling of the nucleic acid.

Variations of MALDI-TOF are available in the art and will be apparent to the skilled artisan.

In another example, a third amplification product is detected by determining the melting temperature of the third amplification product. In one example, a melting temperature assay takes advantage of the different absorption properties of double stranded and single stranded DNA, that is, double stranded DNA absorbs less light than single stranded DNA at a wavelength of 260 nm, as determined by spectrophotometric measurement. This is because heterocyclic rings of nucleotides adsorb light strongly in the ultraviolet range (with a maximum close to 260 nm that is characteristic for each base). However, the adsorption of DNA is approximately 40% less than would be displayed by a mixture of free nucleotides of the same composition. This effect is called hyperchromism and results from interactions between the electron systems of the bases, made possible by their stacking in the parallel array of the double helix. Any departure from the duplex state is immediately reflected by a decline in this effect (that is, by an increase in optical density toward the value characteristic of free bases. Denaturation of double stranded DNA is detectable by this change in optical density. The midpoint of the temperature range over which the strands of DNA separate is called the melting temperature, denoted Tₘ. Moreover, the sequence of a nucleic acid affects the temperature at which the nucleic acid denatures. Accordingly, two sequences amplified with the same primers that differ by even a single nucleotide can be detected by a change in melting temperature.

Melting temperature assays can also make use of a dye that binds to double-stranded nucleic acid and emit a detectable fluorescent signal at a wavelength that is characteristic of the particular dye (see, e.g., Zhang et al., Hepatology 36:723-728, 2002). A dissociation or melting curve can be obtained during or following an amplification reaction by monitoring the nucleic acid dye fluorescence as the reaction temperatures pass through the melting temperature of an amplification product. The dissociation of a double-stranded amplification product is observed as a rapid decrease in fluorescence at the emission wavelength characteristic of the dye. In this manner, it is possible to detect the melting temperature of multiple amplification products comprising different sequences is determined.

### Characterization of an Individual or Group of Individuals

As the present invention is useful for detecting a polymorphism or mutation, the invention has clear application in determining relationships between one or more individuals, isolates of an organism, cultivars of an organism, species or genera. Furthermore, the present invention is useful for identifying an individual, isolate of an organism, cultivar of an organism, species or genus.

For example, the method of the invention is useful for a form of genetic mapping, such as, for example bulked segregant analysis (BSA). In its simplest form this form of analysis uses nucleic acid from a plurality of organisms (preferably, plants) that only differ in one trait (e.g., as a result of mutation or introgression). Nucleic acid from organisms with one phenotype is pooled, as is nucleic acid from organisms with the other phenotype. Using the method of the invention, a region of nucleic acid in which the two pools of nucleic acid differ is determined. Such a method is particularly useful for, for example, mapping of a gene responsible for a monogenic trait or a quantitative trait. Suitable methods for BSA are described, for example, in Wang and Paterson Theor. Appl. Genet. 88:355-361, 1994 and Mackay and Caligari Crop Science 40:626-630, 2000.

Clearly, the present invention contemplates performing a multiplex reaction to identify or characterize an individual, isolate of an organism, cultivar of an organism, species or genus, for example, the detection of a plurality of polymorphisms and/or mutations.

Clearly, the method of the present invention has broad reaching application in any assay that detects one or more polymorphisms or mutations. Accordingly, the present invention is useful for, for example, marker assisted breeding programs (e.g., animal husbandry), gene mapping, the identification of specific strains, races, isolates, serotypes or serogroups of microorganisms, the identification of cultivars, species or genera of plants, and for identification of organisms likely to have a trait of interest.

### Genetic Markers in Plants

Genetic markers are used for a variety of purposes in association with plants. For example, one or more genetic markers is (are) used to identify a specific plant variety. For instance, a plant that is protected by an intellectual property right is characterized to determine one or more genetic markers that are specific to said plant. This then enables simple and rapid characterization of similar plants to determine whether or not an intellectual property right has been infringed.

In another embodiment, the present invention is used to determine a plant that is likely to comprise a trait of interest. Examples of suitable primers for detection of polymorphisms associated with a trait are described herein, or in, for example, Chiapparino et al., Genome. 47:414-420, 2004 (e.g. SNPs in sucrose synthase of Barley); Hayashi et al., Theor Appl Genet. 108:1212-1220, 2004 (blast resistance in rice); and Schwarz et al., J Agric Food Chem. 51:4263-4267, 2003 (SNPs associated with HMW glutenin expression in wheat).

### Genetic Markers in Humans and Animals

The method of the present invention for detecting one or more genetic markers is also useful for, for example, marker assisted breeding of animals and/or to select for those animals with one or more desired traits.

For example, the assay is used to screen animals for enhanced commercial properties, such as, for example, food quality for human consumption. Such an assay is performed to detect one or more markers that is (are) associated with increased marbling in beef. Marbled beef is of commercial importance as consumers in several countries pay a premium price for beef with a high level of marbling. Recently, several markers have been reported that are associated with an increased level of marbling.

For example, Barendse et al., Beef Quality CRC Marbling Symposium, Coffs Harbour pp. 52-57, 2001 describe a SNP in the calpastatin gene (detected using primers comprising the sequence GGGGATGACTACGAGTATGACTG and GTGAAAATCTTGTGGAGGCTGTA.

Furthermore, researchers have reported markers in the leptin gene are associated with an increased marbling score in cattle (Buchanan et al., Genet Sel Evol. 34:105-16, 2002). Accordingly, by producing primers used by Barendse et al. and/or Buchanan et al., tagged with a tag region described herein a multiplex reaction is performed to amplify the respective markers. The amplified nucleic acid is then further amplified using the relevant second set of primers. By subsequently detecting the presence or absence of the described markers cattle with increased marbling scores are identified.

Ciobanu et al., J. Anim. Sci. 82: 2829-2839, 2004 and Chang et al., Vet. J. 165: 157-163, 2003 describe markers useful for determining an increased pork quality from a pig. The marker described by Ciobanu et al., occurs in the calpastatin gene, while the marker described by Chang et al., polymorphism in the desmin gene.

A method described herein according to any embodiment is also applicable to, for example, selecting enhanced race horses (e.g., with enhanced speed and/or endurance), selecting sheep that produce superior wool, or selecting a mammal (e.g., a cow) that produces superior quality milk.

### Diagnostics

As the present invention is useful for the detection of genetic differences, it is particularly useful for the diagnosis of a disease or disorder or the presence of one or more infectious agents in a sample. For example, the method of the invention is useful for detecting a genetic change that is associated with a disease or disorder in a human or a non-human animal.

Exemplary common genetic diseases or disorders in humans associated with a polymorphism or mutation include, for example, cystic fibrosis, sickle cell anemia, β-thalasemia, or muscular dystrophy. Exemplary common diseases in sheep include, for example, Menkes disease or Scrapie. Examples of common genetic diseases in goats include, for example, gynecomastia and anotia-microtia complex. Exemplary genetic diseases in horses include, for example, hyperkalemic periodic paralysis (HYPP), combined immune deficiency syndrome (CID), overo-lethal white syndrome and epitheliogenesis.

The mutations that cause these disorders are now known and, as a consequence, a screen may be developed using the method of the invention to screen for any or all of these disorders in a specific organism.

The present invention is described further in the following non-limiting examples.

### EXAMPLE 1

### Allelic Discrimination by Differential Product Size Using a Pair of Allele Specific Primers Designed to Opposite DNA Strands

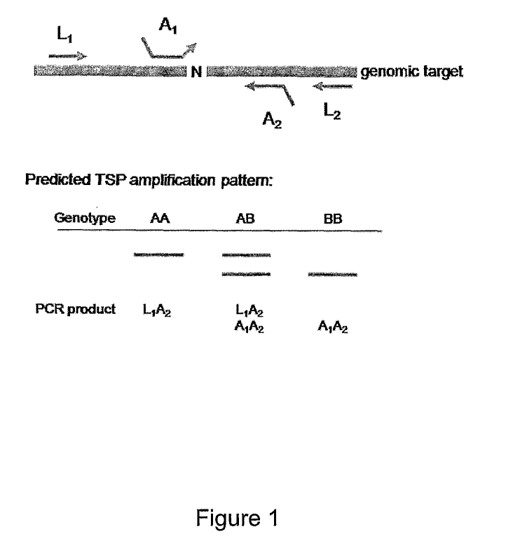

FIG. 1 depicts a method of the present invention for detecting an allele in which allele specificity is conferred by an allele specific (AS) primer that anneals to a locus of interest such that a nucleotide complementary to the allele is positioned at or near the 3′ end of the primer. Accordingly, in the presence of an allele of interest (allele B in FIG. 1) the 3′ end of the primer will anneal to the nucleic acid, however in the presence of an alternate allele (allele A in FIG. 1) the 3′ end of the primer will not anneal or will anneal at a reduced level compared to the level when allele B is present.

As depicted in FIG. 1, the assay is performed using both locus specific (LS) primers (L₁ and L₂) and allele specific (AS) primers (A₁ and A₂). The locus specific primers anneal to nucleic acid in the sample at a first temperature (e.g., from about 63° C. to about 74° C.). These primers amplify the nucleic acid or locus comprising the allele of interest, e.g., in the case of a polyploid organism the LS primers amplify a nucleic acid specific to a genome comprising the allele of interest.

The AS primers comprise a first region that anneals to the nucleic acid comprising the allele of interest at a lower temperature than the annealing temperature of the LS primers. The AS primers also comprise second region, which is a 5′-tail that is not complementary to the nucleic acid comprising the allele. Following amplification of a sequence using an AS primer, the second region is incorporated into the amplification product. Accordingly, the entire AS primer may then anneal to the amplification product at a higher temperature than that of the first region of the AS primer, and preferably at about the same temperature at which a LS primer anneals to a target sequence.

A single PCR is performed using both LS and AS primers. In a first phase, the reaction is performed using an annealing temperature at which the LS primers anneal to a target sequence, however the first region of the AS primers do not substantially anneal to a target sequence. This enriches or amplifies the locus of interest. In a second phase, the reaction is performed using an annealing temperature at which the first region the AS primers anneal to a target sequence. Following several rounds of amplification, the annealing temperature is increased to approximately the same temperature used in the first phase.

In the assay depicted in FIG. 1, the presence of allele B is detected by a PCR fragment that is the product of the AS forward and reverse primer pair (A₁A₂), and hereafter referred to as matched product. The presence of allele A is detected by a PCR product resulting from the LS forward primer and AS reverse primer (L₁A₂), and hereafter referred to as mismatched product. The mismatched product also acts as a positive control against a failed PCR assay. In heterozygous samples, where alleles A and B are present, both matched and mismatched products are amplified.

This assay configuration permits codominant allelic discrimination in a single reaction, and enables the size of the resulting PCR products (matched and mismatched) to be readily adjusted, e.g., within the range of about 60-bp to about 500-bp to suit end-point detection on a variety of size separation matrixes such as agarose gel, and a range of dedicated instruments such as eGENE (eGENE Inc.). Alternatively, allelic discrimination is achieved by end-point or real-time melting analysis using instrumentation such as the RotorGene6000 (Corbett Research), since the matched and mismatched products have different melting temperatures.

### EXAMPLE 2

### Allelic Discrimination by Differential Product Size Using a Pair of Allele Specific Primers Designed to the Same DNA Strand

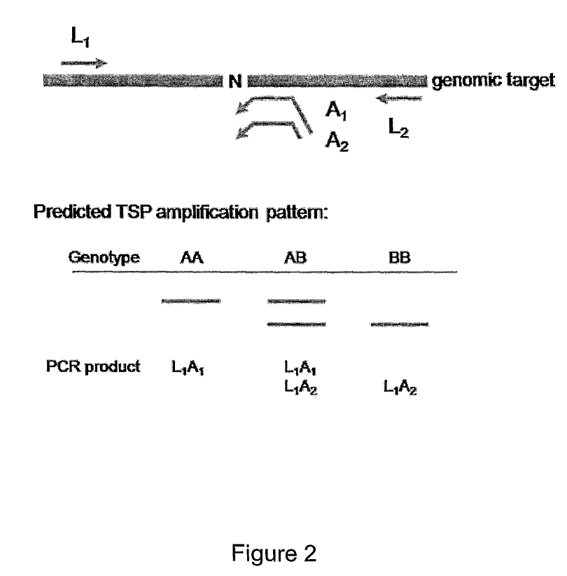

FIG. 2 depicts a method of the present invention for detecting an allele in which specificity for allele A or allele B is conferred by allele specific primers designed to anneal to the same DNA strand. LS and AS primers are produced essentially as described in Example 1, and assays are performed essentially as described in Example 1. In the assay depicted in FIG. 2 both AS primers (A₁ and A₂ in FIG. 2) are designed to anneal to the target locus such that a nucleotide complementary to one allele is positioned at or near the 3′ end of one primer (e.g., A₁), and a nucleotide complementary to the other allele is positioned at or near the 3′ end of the other primer (e.g., A₂). The AS primers differ by having 5′ non-complementary tails of different length.

A single reaction is performed for genotype determination using LS primers L₁ and L₂, and AS primers A₁ and A₂. Depending on the sample genotype, either one or both AS primers anneal to the nucleic acid and amplify to generate an amplification product that is the product of the LS forward primer (L₁) and one AS reverse primer. Unequal length of the PCR products for allele A and allele B enables codominant allelic discrimination using a size separation matrix such as polyacrylamide gel, or a dedicated instrument such as eGENE (eGENE Inc.). Alternatively, allelic discrimination is achieved by end-point or real-time melting analysis using an instrument such as the RotorGene6000 (Corbett Research), since each allele-specific PCR product has a distinct melting temperature that depends on which of the two AS primers is responsible for amplification.

### EXAMPLE 3

### Allelic Discrimination by Differential Product Size Using a Single AS Primer

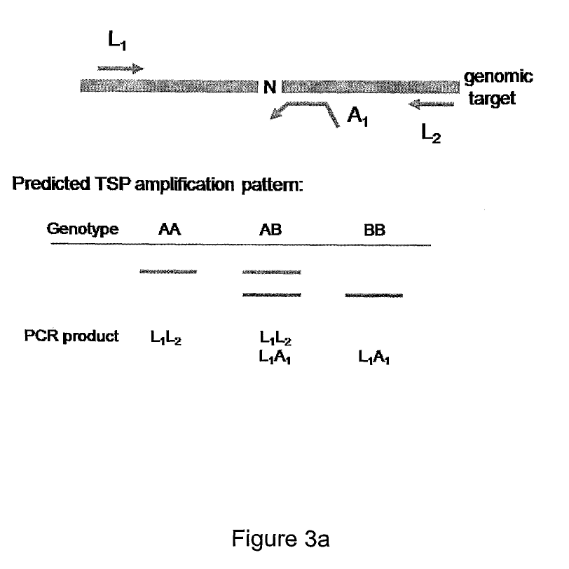

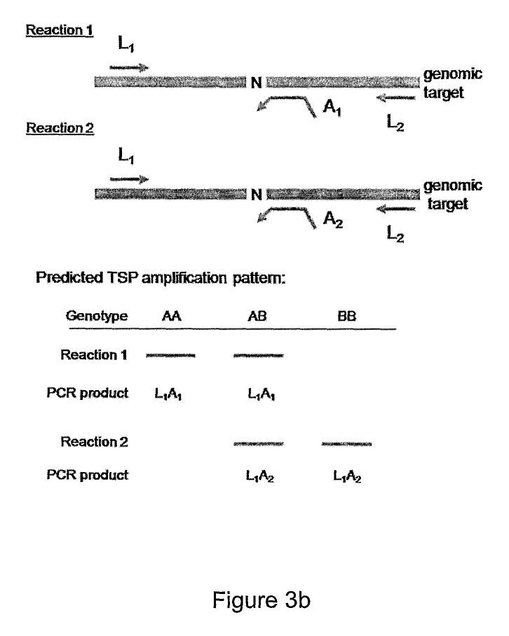

In the assay depicted in FIGS. 3a and 3b, allele specificity is conferred by the AS reverse primer. LS and AS primers are produced essentially as described in Example 1, and assays are performed essentially as described in Example 1. The number of reactions required for genotype determination will be influenced by the size of the PCR fragment amplified by the LS primer pair.

The assay depicted in FIG. 3a is an example in which a PCR fragment amplified by the LS primer pair is relatively short, for example less than about 500-bp. In this case, a single reaction is performed for genotype determination using the LS primers L₁ and L₂ and AS reverse primer A₁. In the assay depicted in FIG. 3a allele specificity is conferred by the AS primer that anneals to a locus of interest such that a nucleotide complementary to the an allele (e.g., the B allele) is positioned at or near the 3′ end of the primer. The presence of allele B is detected by a PCR fragment that is the product of the LS forward primer and AS reverse primers (L₁A₁; matched product), whereas the presence of allele A is detected by a PCR fragment that is the product of the LS primer pair (L₁L₂; mismatched product). Samples heterozygous for the allele are detected by the presence of both matched and mismatched product.

The assay depicted in FIG. 3b is an example of an assay in which a PCR fragment amplified by the LS primer is relatively large, for example, longer than 500-bp. In this situation, two separate reactions are performed for genotype determination, one using the LS primers L₁ and L₂ and AS reverse primer A₁ specific for the allele A (i.e., comprising a sequence complementary to the sequence of allele A), and the other with the LS primers L₁ and L₂ and AS primer reverse primer A₂ specific for allele B (i.e., comprising a sequence complementary to the sequence of allele B). The presence of the allele of interest in each assay is detected by a PCR fragment that is the product of the LS forward primer and AS reverse primer (L₁A₁ or L₁A₂). Samples homozygous at the site of the allele are detected by the presence of matched product in only one reaction, while samples heterozygous at the site of the allele are detected by the presence of matched product in both reactions.

### EXAMPLE 4

### Allelic Discrimination by Differential Product Labeling Using a Pair of AS Primers Designed to the Same DNA Strand

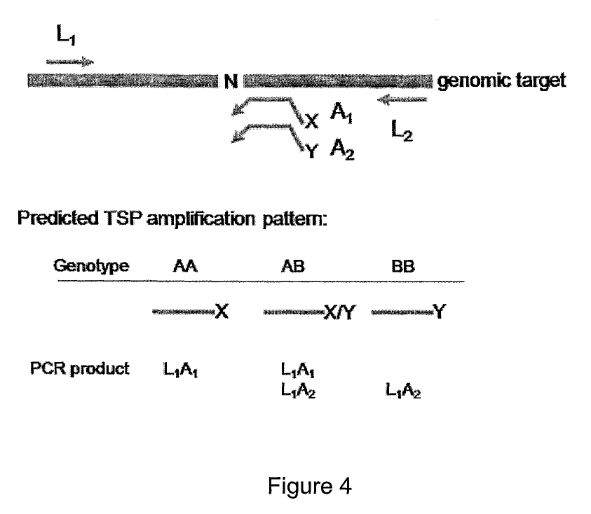

FIG. 4 depicts a method of the present invention for detecting an allele in which specificity for allele A or allele B is conferred by allele specific primers designed to anneal to the same DNA strand. LS and AS primers are produced essentially as described in Example 1, and assays are performed essentially as described in Example 1. In the assay depicted in FIG. 2 both AS primers (A₁ and A₂ in FIG. 2) are designed to anneal to the target locus such that a nucleotide complementary to one allele is positioned at or near the 3′ end of one primer (e.g., A₁), and a nucleotide complementary to the other allele is positioned at or near the 3′ end of the other primer (e.g., A₂). The AS primers differ by having a detectable marker, such as a fluorescent dye attached to their 5′-end.

A single reaction is performed for genotype determination using LS primers L₁ and L₂, and AS primers A₁ and A₂. Depending on the sample genotype, either one or both AS primers anneal to the nucleic acid and amplify to generate an amplification product that is the product of the LS forward primer (L₁) and one AS reverse primer. Differential detection of the detectable marker attached to each AS primer by methods such as fluorescence detection facilitates codominant allelic discrimination.

### EXAMPLE 5

### Allelic Discrimination by Differential Product Detection Using a Pair of AS Primers Designed to Opposite DNA Strands

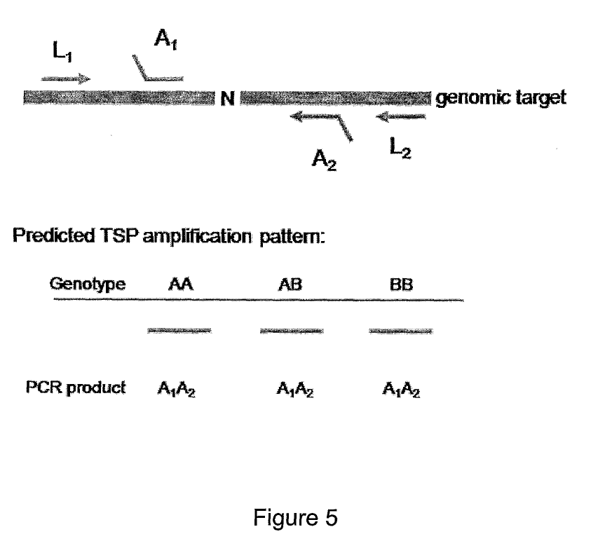

FIG. 5 depicts a method of the present invention for detecting an allele in which allelic discrimination between allele A or allele B is determined using high resolution melting analysis. LS and AS primers are produced similar those described in Example 1, however the AS primers do not anneal to the site of the allele. Rather, the AS primers are adjacent to the allele and, when used in a PCR reaction amplify nucleic acid comprising the allele. A single reaction is performed for genotype determination using the LS primers L₁ and L₂ and AS primers A₁ and A₂, essentially as described in Example 1. Allelic discrimination is determined by end-point and/or real-time high resolution melting analysis due to a difference in the melting temperature between the PCR fragments comprising allele A or allele B, which is the product of the AS primers (A₁A₂; Diagram 5). An advantage of this assay configuration is that the size of the second phase PCR amplification product can be readily adjusted to maximize allele discrimination by high resolution melting analysis.

### EXAMPLE 6

### Allelic Discrimination by Differential Product Detection Using a Single AS Primer

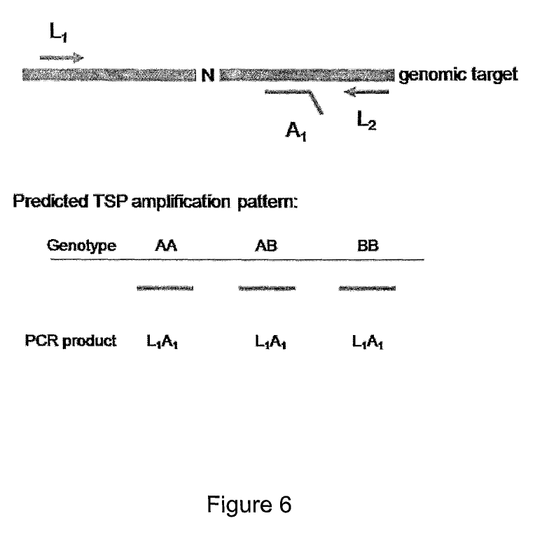

FIG. 6 depicts an alternative method to that described in Example 5 for detecting an allele in which allelic discrimination between allele A or allele B is determined using high resolution melting analysis. A single AS primer and a pair of LS primers are produced similar those described in Example 1, however the AS primer does not anneal to the site of the allele. Rather, the AS primer is adjacent to the allele and, when used in a PCR reaction in combination with a suitable LS primer, amplifies nucleic acid comprising the allele. A single reaction is performed for genotype determination using the LS primers L₁ and L₂ and AS primer A₁. Allelic discrimination is determined by end-point and/or real-time high-resolution melting analysis due to a difference in the melting temperature between the PCR fragments for allele A and allele B, which is the product of the LS forward primer and AS reverse primer (L₁A₁).

### EXAMPLE 7

### Selection and Design of Low-Melting Allele Specific Primers

To minimize the participation of allele specific primers in the first phase of amplification performed using locus specific primers, a series of allele specific primers were tested for amplification yield and specificity under first phase PCR conditions. Specifically, allele specific primers were synthesized for genomic loci harboring known SNPs in barley (Hordeum vulgare) and bread wheat (Triticum aestivum). Each allele specific primer was composed of two parts: a region complementary to sequence flanking the SNP and designed with a melting temperature in the range of 40 to 55° C., and a 5′-tail that was non-complementary to the DNA template, which increased the melting temperature of the AS primer to about 67° C. once the non-complementary tail was incorporated into PCR product. For each target locus, three allele specific primers were synthesized with the complementary region having a melting temperature of 40° C., 45° C., 50° C. and 55° C., respectively. The three allele specific primers for each melting temperature comprise two forward primers and one reverse primer. The two forward primers were designed adjacent to the SNP of interest with a 3′-nucleotide of each primer corresponding to one of the alleles present at the target locus respectively, and a deliberate nucleotide mismatch at the −1, −2 or −3 position from the 3′-terminus, according to Ye et al. Nucl. Acids. Res., 29: e88, 2001. The reverse primer was designed with complete complementarity to the opposite DNA strand at a position 100 to 150-bp downstream of the polymorphism.

The sequences of each of the primers is set forth below:

Primers sequences for the putative gene located on chromosome 5H gene were:

Complementary region melting temperature 40° C.

(SEQ ID NO: 1)

(i) AS forward primer specific for allele A: [symbol]
TCATCACTAGTAAATCTTG

(SEQ ID NO: 2)

(ii) AS forward primer specific for allele B: [symbol]
TCATCACTAGTAAATCTTA

(SEQ ID NO: 3)

(iii) AS reverse primer: [symbol] AGAAAAAGTAATGGT

Complementary region melting temperature 45° C.

(SEQ ID NO: 4)

(i) AS forward primer specific for allele A: [symbol]
AAATCATCACTAGTAAATCTTG

(SEQ ID NO: 5)

(ii) AS forward primer specific for allele B: [symbol]
AAATCATCACTAGTAAATCTTA

(SEQ ID NO: 6)

(iii) AS reverse primer: [symbol] GGGAGAAAAAGTAATGGT

Complementary region melting temperature 50° C.

(SEQ ID NO: 7)

(i) AS forward primer specific for allele A: [symbol]
AGTAAATCATCACTAGTAAATCTTG

(SEQ ID NO: 8)

(ii) AS forward primer specific for allele B: [symbol]
AGTAAATCATCACTAGTAAATCTTA

(SEQ ID NO: 9)

(iii) AS reverse primer: [symbol] TTGTTCTGGACGTTTTCAT

Primers sequences for the nicotinate phosphoribosyltransferase-like gene were:

Complementary region melting temperature 40° C.

(SEQ ID NO: 10)

(i) AS forward primer specific for allele A: [symbol]
GCCGAATCAGTTTAC

(SEQ ID NO: 11)

(ii) AS forward primer specific for allele B: [symbol]
GCCGAATCAGTTTAG

(SEQ ID NO: 12)

(iii) AS reverse primer: [symbol] CTGAATTCACAGGCTG

Complementary region melting temperature 45° C.

(SEQ ID NO: 13)

(i) AS forward primer specific for allele A: [symbol]
CGCCGAATCAGTTTAC

(SEQ ID NO: 14)

(ii) AS forward primer specific for allele B: [symbol]
CGCCGAATCAGTTTAG

(SEQ ID NO: 15)

(iii) AS reverse primer: [symbol] ACTGAATTCACAGGCTG

Complementary region melting temperature 50° C.

(SEQ ID NO: 16)

(i) AS forward primer specific for allele A: [symbol]
GCCGAATCAGTTTAC

(SEQ ID NO: 17)

(ii) AS forward primer specific for allele B: [symbol]
GCCGAATCAGTTTAG

(SEQ ID NO: 18)

(iii) AS reverse primer: [symbol] AACTGAATTCACAGGCTGA

The non-complementary 5′ tail of the AS primers is shown in bold and italics font, the nucleotide corresponding to the SNP is shown in bold, and deliberate mismatches at the −1 or −3 position are underlined.

PCR assays were performed using 1 μM of allele specific forward and reverse primer in a 4 μl reaction mixture containing 0.2 mM dNTP, 1×PCR buffer, 1.5 mM MgCl₂, 100 ng/μl bovine serum albumin Fraction V, between 25 and 50 ng genomic DNA and 0.15 U Platinum Tfi DNA polymerase. Two reactions were performed for each DNA sample, one using an allele specific forward primer specific for one allele, the other with an allele specific forward primer specific for the other allele present at the site of the SNP. Following an initial denaturation step of 2 min at 94° C., PCR was performed for a total of 35 cycles with the profile: 30 s at 92° C., 30 s at 58° C., 2 min at 72° C. The reaction products were separated by electrophoresis in a 1.5% agarose gel and visualized by ethidium bromide staining (Sambrook and Russell 2001, Molecular Cloning: a laboratory manual. Cold Spring Harbor Laboratory Press: Cold Spring Harbor, N.Y.).

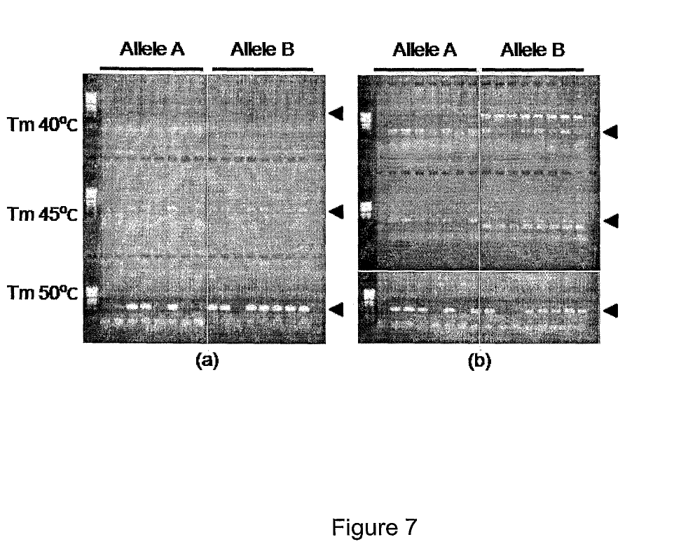

Examination of the PCR specificity and yield revealed that allele specific primers having a complementary region with a melting temperature below 45° C. amplified essentially no PCR product, or product of unexpected size (as shown in FIG. 7). Accordingly, AS primers designed with a complementary region having a melting temperature below 45° C. are expected not to participate significantly in the first phase of TSP amplification. In subsequent experiments, AS primers were designed with the complementary region to have a melting temperature of 40° C.

A deliberate nucleotide mismatch at the −1, −2 or −3 position from the 3′ terminus of allele-specific primers (according to Ye et al. 2000) is not essential to the invention. Nor is this preferred, as it may reduce TSP genotyping accuracy in some cases.

### EXAMPLE 8

### Detection of SNPs Using Temperature Switch PCR and Comparison to Standard PCR Conditions

### Introduction

Without being bound by any theory or mode of action, it is expected that under standard PCR cycling conditions employing a high annealing temperature the LS primers will anneal with high efficiency to the genomic template, resulting in the efficient accumulation of LS product. In contrast, minimal, or no, annealing is expected for the AS primers. However, as the reaction progresses the accumulation of LS product may lead to conditions under which the AS primer can anneal, since the amplification product produced by amplification with LS primers contains sequence complementary to the AS primers. This is expected as the melting temperature of an oligonucleotide primer is related to the concentration of complementary template (Panjkovich and Francisco, Bioinformatics 21: 711-722, 2005). Once LS product has sufficiently accumulated to allow the AS primers to anneal, AS product is produced at high efficiency because of self-amplification. Highly efficient self-amplification occurs, despite the high PCR annealing temperature, because the non-complementary tail of the AS primers is incorporated into the product to provide a much longer region of complementarity. Therefore, the final reaction product is expected to contain a mixture of LS and AS products.

Under cycling conditions of the method of the present invention, it is also expected that LS product will accumulate with high efficiency at the high PCR annealing temperature used in the first phase of the reaction. However, a lowering of the annealing temperature after 15 cycles of first phase amplification enables the AS primers to participate in PCR amplification before their participation might otherwise be expected. Efficient annealing of the AS primers to the enriched target sequence (amplification product produced by amplification with LS primers) at the second phase annealing temperature allows for highly efficient self-amplification of AS product in subsequent cycles due to incorporation of the non-complementary 5′-tail. Accordingly, the accumulation of AS product during the second phase of TSP amplification is expected to out-compete the accumulation of LS product, resulting in a predominance of AS product.

To demonstrate the TSP mechanism, the accumulation of amplicons produced from amplifications with LS or AS primers was monitored. In the assays, AS primers were designed to opposite DNA strands (see FIG. 1). Assays were performed using samples with known zygosity and different combinations of the four LS and AS primers to show the contribution of each primer to the accumulation of the expected PCR products. For comparison, each reaction was also performed under standard PCR cycling conditions with a high annealing temperature and the same number of cycles for amplification.

PCR assays were performed using 0.1 μM of LS primer and 1 μM of AS primer in a 4 μl reaction mixture containing 0.2 mM dNTP, 1×PCR buffer, 1.5 mM MgCl₂, 100 ng/μl bovine serum albumin Fraction V, between 25 and 50 ng genomic DNA and 0.15 U Platinum Tfi DNA polymerase. The composition of LS and AS primer in each reaction is described in Table 1. Assays performed using standard PCR cycling conditions were performed with an initial denaturation step of 2 min at 94□C, followed by 35 cycles of 30 s at 92° C., 30 s at 58° C., 2 min at 72° C.

Reaction products were separated by electrophoresis in a 1.5% agarose gel and visualized by ethidium bromide staining.

For the assay of the invention, amplification reactions were performed using the following reaction mixture: 0.2 mM dNTP, 1×PCR buffer (16 mM (NH₄)₂SO₄, 0.01% Tween-20, 100 mM Tris-HCl, and pH 8.3), 1.5 mM MgCl₂, 100 ng/μl bovine serum albumin Fraction V (Sigma Aldrich), 0.1 μM each of each locus specific forward and reverse primer, 1 μM each of each allele specific forward and reverse primer, between 25 and 50 ng genomic DNA and 0.15 U Platinum Tfi DNA polymerase (Invitrogen) (total reaction volume 4 μl). Amplification reactions were performed under the following conditions:
(i) initial denaturation, 2 min at 94° C.;
(ii) 35 cycles with the profile: 30 s at 92° C., 30 s at 58° C., 2 min at 72° C. for 15 cycles (hereafter referred to as the first reaction phase);
(iii) 5 cycles were with 10 sec at 92° C. and 30 sec at 35° C.;
(iv) 15 cycles with 10 sec at 92° C., 30 sec at 58° C. (hereafter referred to as the second reaction phase);
(v) 10 min at 72° C.; and
(vi) indefinite hold at 15° C.

Reaction products were separated by electrophoresis in a 1.5% agarose gel and visualized by ethidium bromide staining.

TABLE 1
LS and AS primers present in each set of reactions.

| Reaction | Primer Combination |
|---|---|
| 1 | L₁ L₂ |
| 2 | A₁ₐ A₂ |
| 3 | A_{1b} A₂ |
| 4 | L₁ A₁ₐ A₂ |
| 5 | L₁ A_{1b} A₂ |
| 6 | L₂ A₁ₐ A₂ |
| 7 | L₂ A_{1b} A₂ |
| 8 | L₁ L₂ A₁ₐ |
| 9 | L₁ L₂ A_{1b} |
| 10 | L₁ L₂ A₂ |
| 11 | L₁ L₂ A₁ₐ A₂ |
| 12 | L₁ L₂ A_{1b} A₂ |
| 13 | L₁ A₂ |
| 14 | L₂ A₁ₐ |
| 15 | L₂ A_{1b} |

where,
L₁ is LS forward primer
L₂ is LS reverse primer
A₁ₐ is AS forward primer specific for allele A
A_{1b} is AS forward primer specific for allele B
A₂ is AS reverse primer
as depicted in FIG. 1 and described in Example 1

Primers used in one example of the assay are as follows:

(i) LS forward primer, L₁:
TGTGTCTGAACTTGCATTTGATGACG
(ii) LS reverse primer, L₂:
CCTCTCTTTGTGCTCTCAACTTGTCCA
(iii) AS forward primer specific for allele A,
A₁ₐ: [symbol] TCATCACTAGTAAATCTTG
(iv) AS forward primer specific for allele B, A_{1b}: [symbol]
TCATCACTAGTAAATCTTA
(v) AS reverse primer, A₂: [symbol]
AGAAAAAGTAATGGT

The non-complementary 5′ tail of the AS primers is in highlighted in bold italic font, and deliberate mismatches at the −3 position are underlined.

If the TSP assay mechanism functions as predicted, correct genotype determination should only be achieved for reactions performed under TSP cycling conditions in the presence of all four LS and AS primers (reactions 11 and 12, Table 1). All other reactions should produce no amplification product, or PCR product the size of which depends on the interactions among the primers present.

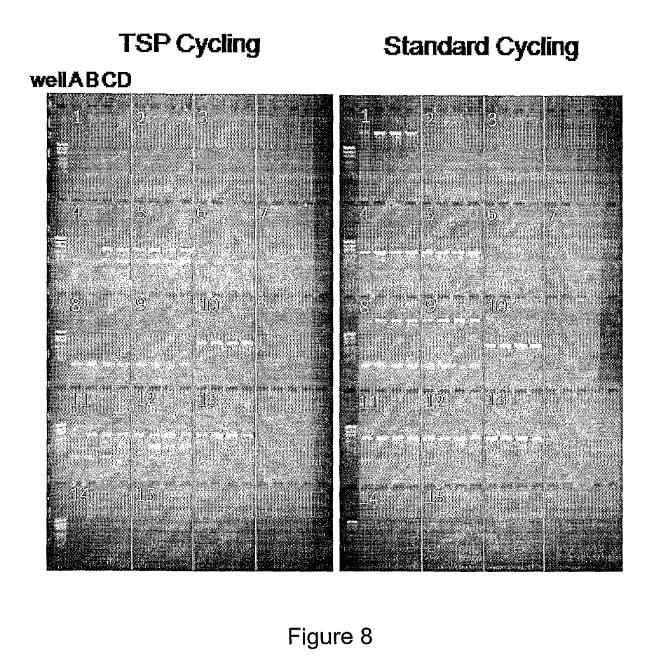

Inspection of the PCR fragments amplified across multiple DNA samples and genomic loci revealed that genotype determination was more accurate for assays of the invention in which the four LS and AS primers were present (reactions 11 and 12, FIG. 8). All other primer combinations tested under TSP cycling conditions gave no amplification, incorrect genotypes or inconsistent genotyping accuracy. For example, assays performed using only the LS forward primer L₁ and the pair of AS primers A₁ and A₂ often produced the expected genotype (reactions 4 and 5, FIG. 8), but were not as reliable for genotype determination across larger numbers of samples. Correct genotype determination was not achieved for any of the reactions performed using standard PCR cycling conditions (FIG. 8). The results demonstrate that accurate genotype determination for the assay format tested was achieved best under cycling conditions of the assay of the invention, in the presence of all four LS and AS primers. These results affirm that the reaction mechanism of the assay of the invention involves sequential enrichment of a target sequence harboring the SNP by the LS primers L₁ and L₂, followed by nested amplification of the interrogated allele by the AS primers A₁ and A₂.

The presence of mismatched product resulting from the LS forward primer L₁ and AS reverse primer A₂ in samples homozygous for the reference allele, in which only matched product resulting from the AS forward and reverse primer pair A₁ and A₂ was expected, indicates that interactions among primers affect both the yield and specificity of TSP reactions (reactions 11 and 12, FIG. 8). In general, the amount of mismatched product observed in samples homozygous for the reference allele varied between different genomic loci, and ranged from almost absent (i.e. only matched product was observed) to having sufficient yield to suggest that the sample was heterozygous. It is likely that the presence of mismatched product in these sample results from a destabilization of the annealing efficiency of the AS forward primer A₁, compared to the AS reverse primer A₂, due to the presence of the secondary mismatch at the −1 or −2 position from the 3′-terminus. Primer destabilization may lead to more efficient formation of mismatched product during the initial cycles of second phase TSP amplification. Assays to ensure correct genotype are described below.

### EXAMPLE 9

### Separate TSP Assays to Detect Each Form of an Allele

Assays were configured for allelic discrimination by differential product size using a pair of AS primers designed to opposite DNA strands (as shown in Diagram 1). The assays were performed in barley (Hordeum vulgare) using samples with known zygosity, using the following assay reaction mixture: 0.2 mM dNTP, 1×PCR buffer (16 mM (NH₄)₂SO₄, 0.01% Tween-20, 100 mM Tris-HCl, and pH 8.3), 1.5 mM MgCl₂, 100 ng/μl bovine serum albumin Fraction V (Sigma Aldrich), 0.1 μM each of each locus specific forward and reverse primer, 1 μM each of each allele specific forward and reverse primer, between 25 and 50 ng genomic DNA and 0.15 U Platinum Tfi DNA polymerase (Invitrogen) (total reaction volume 4 μl). Amplification reactions were performed under the following conditions:
(i) initial denaturation, 2 min at 94° C.;
(ii) 35 cycles with the profile: 30 s at 92° C., 30 s at 58° C., 2 min at 72° C. for 15 cycles (hereafter referred to as the first reaction phase);
(iii) 5 cycles were with 10 sec at 92° C. and 30 sec at 35° C.;
(iv) 15 cycles with 10 sec at 92° C., 30 sec at 58° C. (hereafter referred to as the second reaction phase);
(v) 10 min at 72° C.; and
(vi) indefinite hold at 15° C.

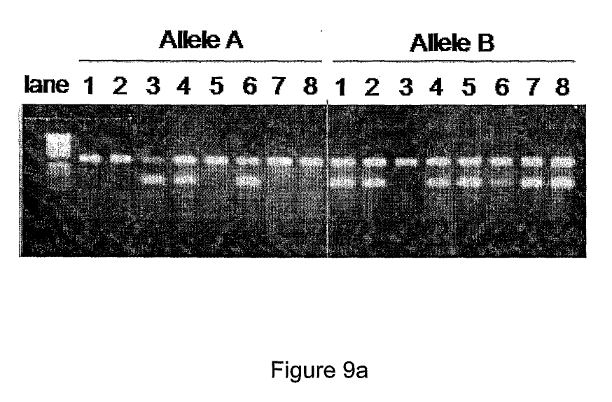

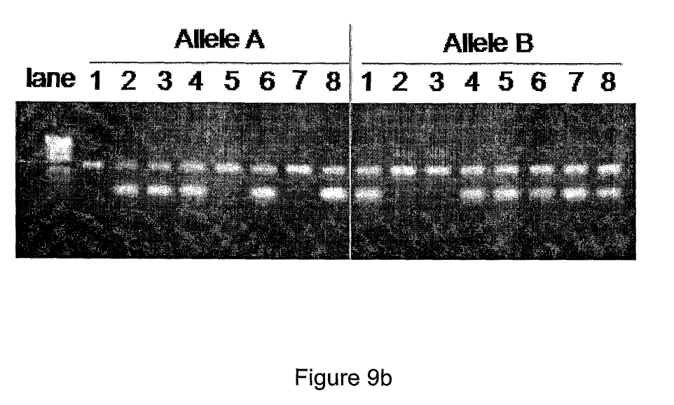

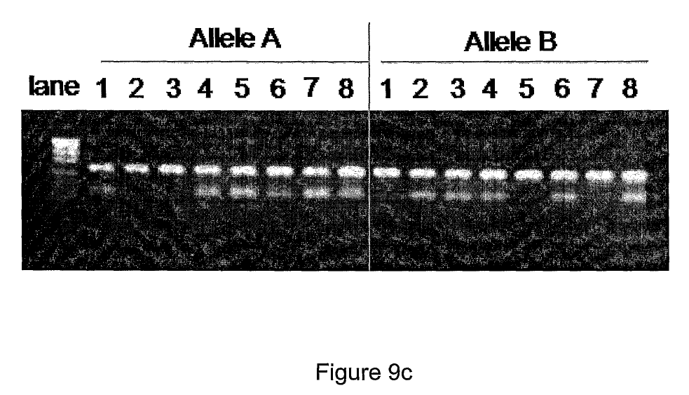

AS primers used have a complementary region melting temperature of 40° C. Two reactions were performed for each sample, one with AS forward primers specific for allele A, and the other with AS forward primer specific for allele B. Primers were designed to assay SNPs in putative gene located on chromosome 5H (FIG. 9a), and a nicotinate phosphoribosyltransferase-like gene (FIGS. 9b and 9c). Primer sequences are as follows:

Primers sequences for the putative gene located on chromosome 5H gene were:

(SEQ ID NO: 24)

(i) LS forward primer, L₁:
TGTGTCTGAACTTGCATTTGATGACG

(SEQ ID NO: 25)

(ii) LS reverse primer, L₂:
CCTCTCTTTGTGCTCTCAACTTGTCCA

(SEQ ID NO: 26)

(iii) AS forward primer specific for allele A,
A₁ₐ: [symbol] TCATCACTAGTAAATCTTG

(SEQ ID NO: 27)

(iv) AS forward primer specific for allele B, A_{1b}: [symbol]
TCATCACTAGTAAATCTTA

(SEQ ID NO: 28)

(v) AS reverse primer: [symbol] AGAAAAAGTAATGGT

Primers sequences for the nicotinate phosphoribosyltransferase-like gene (FIG. 9b) were:

(SEQ ID NO: 29)

(i) LS forward primer, L₁: CTACTGGAAGGCCGGCAAGC

(SEQ ID NO: 30)

(ii) LS reverse primer, L₂:
CGCATAAACCTCAACATCTGAGCA

(SEQ ID NO: 31)

(iii) AS forward primer specific for allele A,
A₁ₐ: [symbol] GCCGAATCAGTTTAC

(SEQ ID NO: 32)

(iv) AS forward primer specific for allele B, A_{1b}: [symbol]
GCCGAATCAGTTTAG

(SEQ ID NO: 33)

(v) AS reverse primer: [symbol] TGAATTCACAGGCTG

Primers sequences for the nicotinate phosphoribosyltransferase-like gene (FIG. 9c) were:

(SEQ ID NO: 34)
(i) LS forward primer, L₁: CTACTGGAAGGCCGGCAAGC
(SEQ ID NO: 35)
(ii) LS reverse primer, L₂:
CGCATAAACCTCAACATCTGAGCA
(SEQ ID NO: 36)
(iii) AS forward primer specific for allele A,
A₁ₐ: [symbol] CGCGTGACAACTAAAATTATACA
(SEQ ID NO: 37)
(iv) AS forward primer specific for allele B, A_{1b}: [symbol]
CGCGTGACAACTAAAATTATACT
(SEQ ID NO: 38)
(v) AS reverse primer: [symbol] TCGCTCATACAAGTGGAA

The non-complementary 5′ tail of the AS primers is in highlighted in bold italic font, and deliberate mismatches at the −1, −2 or −3 position are underlined.

As shown in FIGS. 9a-9c one manner in which to achieve correct genotype determination for genomic loci producing mismatched product in samples homozygous for a reference allele is to perform two assays, one specific for the reference allele and the other specific for the alternate allele.

### EXAMPLE 10

### Altering Melting Temperature of Allele Specific Primers to Normalize Annealing Efficiency

Assays were configured for allelic discrimination by differential product size using a pair of AS primers designed to opposite DNA strands (see FIG. 1 and Example 1). The assay was performed using genomic DNA from bread wheat (Triticum aestivum) using samples with known zygosity. The AS forward and reverse primers A₁ and A₂ had complementary region melting temperatures of 50° C. and 40° C., respectively. Two reactions were performed for each sample, one using the AS forward primer specific for allele A, and the other using AS forward primer specific for allele B. Assay conditions were essentially as described in Example 9. Primers were designed to assay a SNP located in a putative nodulin gene on the chromosome 3B.

Primer sequences for the putative nodulin gene were as follows:

(SEQ ID NO: 39)

(i) LS forward primer, L₁: TACTTCCTCGAGAAGTACGCCG

(SEQ ID NO: 40)

(ii) LS reverse primer, L₂: GTAGAGCGTGATCACCGTGG

(SEQ ID NO: 41)

(iii) AS forward primer specific for allele A,
A₁ₐ: [symbol] GCTTCTGCCAGTCTC

(SEQ ID NO: 42)

(iv) AS forward primer specific for allele B, A_{1b}: [symbol]
GCTTCTGCCAGTGAG

(SEQ ID NO: 43)

(v) AS reverse primer, A₂: [symbol] CAGCGAGAAGGTGAG

The non-complementary 5′ tail of the AS primers is in highlighted in red font, and deliberate mismatches at the −2 or −3 positions are underlined.

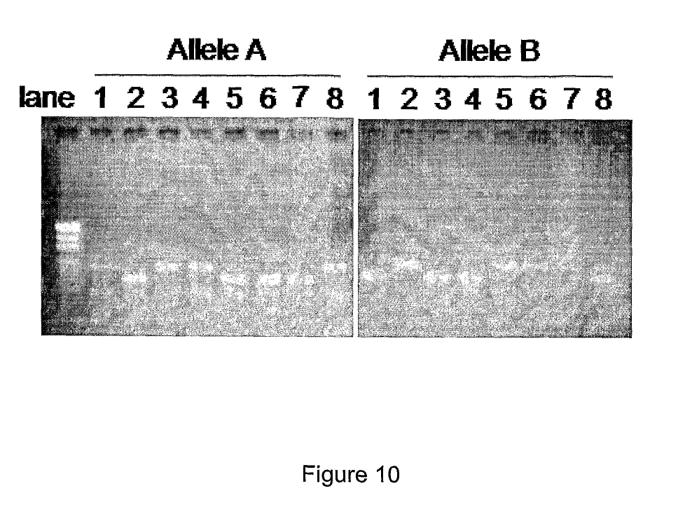

As shown in FIG. 10, increasing the melting temperature of the complementary region in the AS forward primer A₁, relative to the melting temperature of the AS reverse primer A₂, normalizes the annealing efficiency of the AS primer pair during the initial cycles of second phase of TSP amplification. Such normalization facilitates achieve correct genotype determination for genomic loci producing mismatched product in samples.

### EXAMPLE 11

### Discrimination by Differential Product Detection

For some assay configurations, such as allelic discrimination by differential product detection (see FIGS. 5 and 6 and Examples 5 and 6), the capture of sequence variation within the second phase PCR amplification product eliminates the requirement for AS primers to contain mismatched nucleotides that can cause primer annealing destabilization.

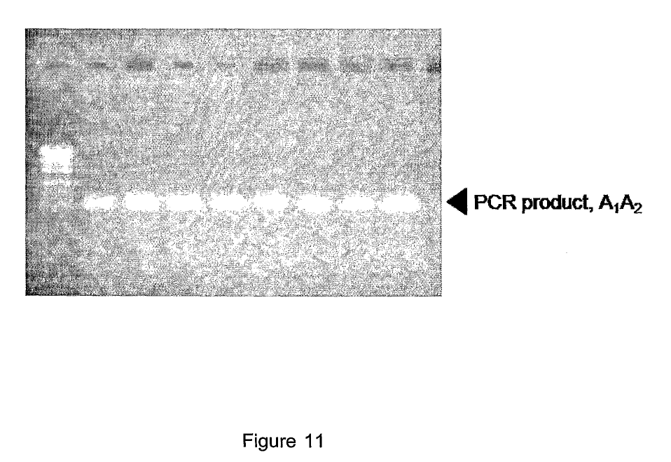

Results of such an assay are shown in FIG. 11. The assay was performed using genomic DNA from barley (Hordeum vulgare) using samples with known zygosity. The AS primers have a complementary region melting temperature of 40° C. Assay conditions were essentially as described in Example 9. Primers were designed to assay a SNP in a nicotinate phosphoribosyltransferase-like gene as follows

(SEQ ID NO: 44)

(i) LS forward primer, L₁: CTACTGGAAGGCCGGCAAGC

(SEQ ID NO: 45)

(ii) LS reverse primer, L₂:
CGCATAAACCTCAACATCTGAGCA

(SEQ ID NO: 46)

(iii) AS forward primer, A₁: [symbol] GCCGAATCAGTTTG

(SEQ ID NO: 47)

(iv) AS reverse primer, A₂: [symbol] GAATTCACAGGCTG

The non-complementary 5′ tail of the AS primers is in highlighted in bold italic font.

These assay configurations result in the efficient accumulation of the expected PCR fragment (as shown in FIG. 10).

### EXAMPLE 12

### Blinded Analysis of Results

To test the sensitivity and accuracy of the assay of the invention for actual genotype determination, a blinded study was performed using F₄ progeny derived from crosses between the barley lines Chebec and Harrington, Amagi Nijo and WI2585, and Haruna Nijo and Galleon. Assays were developed for 28 SNPs identified by Sanger sequencing in 23 genes located in a region on chromosome 2H containing a frost tolerance QTL, and chromosome 5H containing a malting quality QTL. The mapping populations, each comprising about 250 individuals, were screened independently for each SNP using cleaved amplified polymorphism (CAP) assays (Minamiyama et al. Plant Breeding 124: 288-291, 2005) and the assay of the present invention. For each SNP, two separate assays of the invention were performed, one specific for allele A, and the other specific for allele B. Complete concordance between the two genotyping methods across all assays demonstrated that the assay of the present invention achieves high genotyping accuracy.

### EXAMPLE 13

### Further Evidence of Biphasic Nature of TSP Amplification

To further demonstrate the reaction mechanism for biphasic PCR amplification of TSP genotyping products, real-time PCR assays were performed to monitor the accumulation of LS and AS product in TSP assays configured for allelic discrimination by differential product size using a pair of AS primers designed to opposite DNA strands e.g., as shown in FIG. 1. TSP assays were performed using DNA samples with known zygosity and different combinations of the four LS and AS primers (Table S1) to show the contribution of each primer to the accumulation of the expected PCR products.

Real-time PCR assays were performed on a RotorGene6000 thermocycler (Corbett Research) using SYBR Green detection in a 12 μl reaction mixture containing 0.2 mM dNTP, 1×PCR buffer (16 mM (NH₄)₂SO₂, 0.01% Tween-20, 100 mM Tris-HCl, pH 8.3), 1.5 mM MgCl₂, 100 ng/μl bovine serum albumin Fraction V, 0.1 μM LS primer, 0.5 μM AS primer, 0.45 U Platinum Tfi DNA polymerase (Invitrogen) and 20 ng genomic DNA. Following an initial denaturation step of 2 min at 94° C. to heat activate the DNA polymerase, PCR was performed for a total of 65 cycles with the profile: 30 s at 92° C., 30 s at 58° C., 2 min at 72° C. for 15 cycles (hereafter referred to as the first reaction phase). The next five cycles were with 10 s at 92° C., 30 s at 45° C., followed by 45 cycles with 30 s at 92° C., 30 s at 53° C., 5 s at 72° C. (hereafter referred to as the second reaction phase). The accumulation of reaction products was monitored during each PCR cycle by measuring changes in SYBR Green fluorescence.

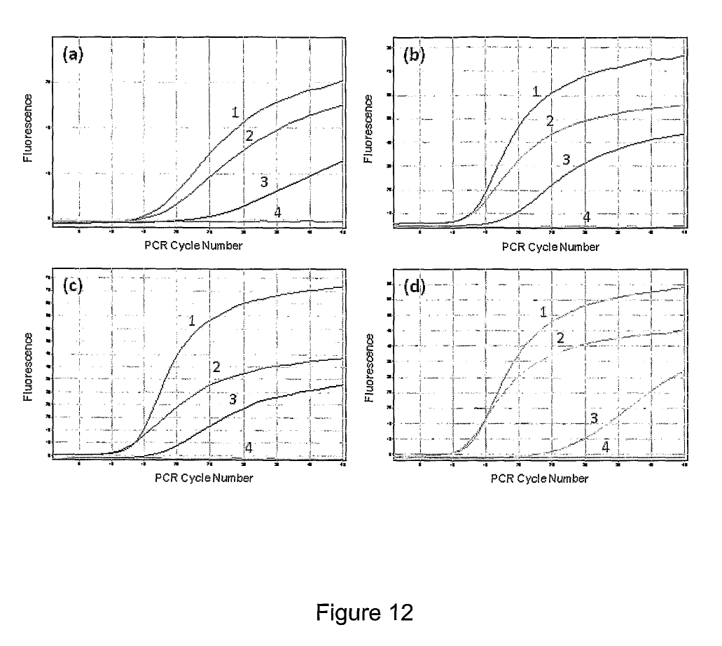

Primer combinations used in each assay were as described in the legend to FIG. 12.

Primers sequences for gene encoding a putative Rieske Fe—S precursor protein:

(SEQ ID NO: 78)

LS forward primer, L1: CGAGGATTGGCTCAAGACGC;

(SEQ ID NO: 79)

LS reverse primer, L2: GCAGCGTTCTTAGGACTGGCA;

(SEQ ID NO: 80)

AS forward primer, A1: CGAATGGATTCTTCAGAAAAG;

(SEQ ID NO: 81)

AS reverse primer, A2: GCGTTCCTCTGCCCTTG.

Primers sequences for gene encoding fructose-6-phosphate 2-kinase:

(SEQ ID NO: 82)

LS forward primer, L1: GCGTCGCAAAGACAAGCTGA;

(SEQ ID NO: 83)

LS reverse primer, L2: CCGCAGGCGAACCTTTACAT;

(SEQ ID NO: 84)

AS forward primer, A1: CGTGCATACTGCACAAAAT;

(SEQ ID NO: 85)

AS reverse primer, A2: GCACCTCATAAAGAATGGTTC.

Primers sequences for gene encoding an unnamed protein product from rice:

(SEQ ID NO: 86)

LS forward primer, L1: GAAGTCGACGCTGATGGCAA;

(SEQ ID NO: 87)

LS reverse primer, L2: TCGTGCGATCCGTTTTAGCA;

(SEQ ID NO: 88)

AS forward primer, A1: GGGTCTTCGGAGCACGA;

(SEQ ID NO: 89)

AS reverse primer, A2: GCAATCTCGGCGAGAAG.

Primers sequences for gene encoding cytosolic aldehyde dehydrogenase:

(SEQ ID NO: 90)

LS forward primer, L1: CGGAGATCCTTTCAACCCGA;

(SEQ ID NO: 91)

LS reverse primer, L2: TCGGATGTCCGTCCAGATCA;

(SEQ ID NO: 92)

AS forward primer, A1: GGCATTTTGTAACATGTTCAG;

(SEQ ID NO: 93)

AS reverse primer, A2: CGGTCGGTAAGAGCGAAG.

The non-complementary 5′ tail of the AS primers is underlined in each case.

Data shown in FIG. 12 indicate that the TSP assay mechanism functions as predicted, because accumulation of PCR product occurs earlier in reactions containing LS primer (Reactions 1 and 2, FIG. 12) by virtue of only those primers efficiently hybridizing to genomic template at the high PCR annealing temperature used in the first phase of the reaction. As expected, the accumulation of PCR product was more rapid for reactions containing LS primer (Reactions 1 and 2, FIG. 12), compared to reactions containing only AS primer (Reaction 3, FIG. 12). These data indicate that the amplification of PCR product in reactions with only AS primer is efficient only after the PCR annealing temperature is lowered in the second stage of the reaction. These results demonstrate an effective partitioning in TSP assays of the participation of LS and AS primers in the first and second reaction stages, respectively.

Furthermore, the real-time PCR data demonstrates an efficient transition from the amplification of LS product to the accumulation of AS product in the second phase of the reaction. Reactions containing both LS and AS primers (Reaction 2, FIG. 12) consistently had lower relative fluorescence at each PCR cycle, compared to reactions containing only LS primer (Reaction 1, FIG. 12). Reduced fluorescence corresponds to the transition from amplification of LS product to that of AS product, and is observed because AS product is significantly shorter than LS product (typically by more than 100-bp). SYBR Green dye binds only to double-stranded DNA, producing an increase in fluorescence that is proportional to both the total amount of, and length of the PCR product.

Thus, data in FIG. 12 demonstrate the efficient annealing of AS primers to the enriched target sequence (LS product) at the second phase annealing temperature, allowing for highly efficient self-amplification of AS product in subsequent cycles due to incorporation of the non-complementary 5′-tail, and therefore, out-competing of the accumulation of LS product.

### EXAMPLE 14

### TSP Amplification to Discriminate Alleles in a Methylenetetrahydrofolate Reductase (MTHFR) Gene of Humans

This example demonstrates the application of the method of the present invention to discriminating between alleles of a clinically significant diseases and disorders in humans. The methylenetetrahydrofolate reductase gene (MTHFR; GenBank Accession No. NM 005957) is located on human chromosome 1 p36.3. The gene encodes the enzyme, methylenetetrahydrofolate reductase (EC 1.5.1.20), which catalyzes the conversion of 5,10-methylenetetrahydrofolate to 5-methyltetrahydrofolate, a co-substrate for homocysteine remethylation to methionine. The most widely studies polymorphism in this gene (C677T; rs1801133) results in an alanine to valine substitution at position 222 resulting in a thermolable enzyme with reduced activity that has been implicated in folic acid deficiency. This polymorphism has also been associated with neural tube defects, arterial and venous thrombosis, cardiovascular disease and schizophrenia. Homozygous mutant (677TT) individuals are at a decreased risk of certain leukemias and colon cancer. The rs1801133 SNP is presented in the following sequence:

(SEQ ID NO: 94)

TGAAGGAGAAGGTGTCTGCGGGAG[C/T]CGATTTCATCATCACGCAG
CTTT.

To produce template nucleic acids for TSP amplification, 48 genomic DNA samples were obtained and purified from human brain tissue and concentration adjusted to 20 ng/μl.

Primers were as follows:

(SEQ ID NO: 95)

F-LS.MTHFR: TCTTCATCCCTCGCCTTGAA;

(SEQ ID NO: 96)

R-LS.MTHFR: GCCTGCCGTTTTCTCCTCTT;

(SEQ ID NO: 97)

F-AS.MTHFR_REF: GCGTGTCTGCGGGAGC;
and

(SEQ ID NO: 98)

R-AS.MTHFR CGGATGGGGCAAGTGAT,

wherein F-LS and R-LS are locus-specific primers LS1 and LS2, respectively, and wherein F-AS and R-AS indicate allele-specific primers AS1 and AS2, respectively.

The non-complementary 5′ tail of the AS primers is underlined in each case.

Amplifications were performed in 96-well PCR plates in a 15 μl final reaction volume consisting of 20 ng genomic DNA, 1×PCR buffer (Invitrogen), 100 ng/μl bovine serum albumin Fraction V (Sigma-Aldrich), 1.5 mM MgCl₂, 0.2 mM of each dNTP, 0.1 μM each locus-specific primer, 0.5 μM each allele-specific primer and 0.375 U Taq DNA polymerase (Invitrogen).

Thermal amplification was performed in a 225-PTC thermal cycler (MJ Research, Bio-Rad) using the following conditions: 94° C. for 3 mins, followed by 15 cycles of 92° C. for 30 s, 58° C. for 30 s and 72° C. for 60 s, 5 cycles consisting of 92° C. for 10 s and 45° C. for 30 s and 15 cycles consisting of 92° C. for 10 s, 53° C. for 30 s and 72° C. for 5 s with a final extension step of 72° C. for 10 mins.

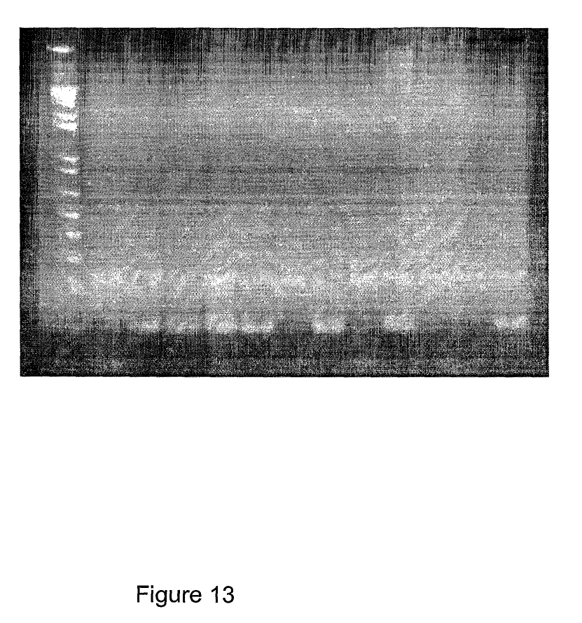

Data presented in FIG. 13 indicate specific amplification of both alleles which are successfully resolved using 2% (w/v) agarose by virtue of the smaller size and more rapid mobility of the 677T allele relative to the 677C allele. Homozygotes for both alleles, and heterozygotes, are readily identified using this method.

### EXAMPLE 15

### TSP Amplification to Identify HSV and Discriminate Between HSV-1 and HSV-2

This example demonstrates the application of the method of the present invention to detect HSV in a sample and discriminate between HSV-1 and HSV-2.

Herpes simplex virus is a viral infectious agent of humans. There are two infectious forms of the virus: herpes simplex 1 (HSV-1) and herpes simplex 2 (HSV-2). HSV-1 infection is contracted through direct contact with an active lesion or bodily fluid of an infected person. It is generally acquired during childhood and adolescence, and primarily affects the face and mouth. HSV-2 infection is a sexually-transmitted disease and primarily affects the genitalia. Although gential herpes is largely caused by HSV-2, gential HSV-1 infections are common. Similarly, cases of orofacial herpes caused by infection of HSV-2 are known. Diagnostic tests to distinguish HSV-1 and HSV-2 infection types are therefore important.

PCR primers for the TSP assay are based on the nucleotide sequence of the virion glycoprotein B (UL27) gene of HSV-1 (Genbank Accession No. AB252863) and HSV-2 (Genbank Accession No. AB442016). The forward locus-specific (F-LS) and reverse locus-specific (R-LS) primers are designed to nucleic acid sequence conserved between HSV-1 and HSV-2. A single forward allele-specific (F-AS) primer is designed to assay a single nucleotide polymorphism (SNP) distinguishing HSV-1 and HSV-2.

Primers are as follows:

(SEQ ID NO: 99)

F-LS.HSV: GCCACCGCTACTCCCAGTTT;

(SEQ ID NO: 100)

R-LS.HSV: CCTCCTCGACGATGCAGTT;

(SEQ ID NO: 101)

F-AS.HSV-1: CACGACATGGAGCTGAAA,

wherein F-LS and R-LS are locus-specific primers LS1 and LS2, respectively, and wherein F-AS indicates an allele-specific primer AS1 specific for HSV-1.

The non-complementary 5′ tail of the AS primers is underlined in the allele-specific primer, and nucleotides specific for HSV-1 are shown in bold font.

Amplifications are performed in 96-well PCR plates in a 15 μl final reaction volume consisting of 20 ng DNA, 1×PCR buffer (Invitrogen), 100 ng/μl bovine serum albumin Fraction V (Sigma-Aldrich), 1.5 mM MgCl₂, 0.2 mM of each dNTP, 0.1 μM each locus-specific primer, 0.5 μM allele-specific primer and 0.375 U Taq DNA polymerase (Invitrogen).

Thermal amplification is performed in a 225-PTC thermal cycler (MJ Research, Bio-Rad) using the following conditions: 94° C. for 3 mins, followed by 15 cycles of 92° C. for 30 s, 58° C. for 30 s and 72° C. for 60 s, 5 cycles consisting of 92° C. for 10 s and 45° C. for 30 s and 15 cycles consisting of 92° C. for 10 s, 53° C. for 30 s and 72° C. for 5 s with a final extension step of 72° C. for 10 mins.

HSV-1 in a sample produces a 139-bp amplification product of the F-AS and R-LS primers, whereas HSV-2 in a sample produces a 300-bp amplification product of the same F-LS and R-LS primers. Accordingly, the presence of both the 139 and 300-bp amplification products indicates the presence of both types of herpes simplex virus. The absence of PCR product indicates the absence of both HSV-1 and HSV-2.

### EXAMPLE 16

### TSP Amplification to Identify HSV and Discriminate Between HSV-1 Strains

This example demonstrates the application of the method of the present invention to detect HSV-1 in a sample and discriminate between HSV-1 strains MP-S and gC-39-R6.

PCR primers for the TSP assay are based on the nucleotide sequence of the virion glycoprotein B (UL27) gene of HSV-1 (Genbank Accession No. EF177454) and HSV-2 (Genbank Accession No. EF177453). The forward locus-specific (F-LS) and reverse locus-specific (R-LS) primers are designed to nucleic acid sequence specific to HSV-1 such that HSV-2 sequences are not amplified. A single forward allele-specific (F-AS) primer is designed to assay a single nucleotide polymorphism (SNP) distinguishing HSV-1 strain MP-S from HSV-1 gC-39-R6.

Primers are as follows:

(SEQ ID NO: 102)

F-LS.HSV: CAGCGCCATGTCAACGATATGT;

(SEQ ID NO: 103)

R-LS.HSV: CGCATCGAGTTTTGGACGAT;

(SEQ ID NO: 104)

F-AS.HSV (MP-S): TGCATCGCCTCGGC,

wherein F-LS and R-LS are locus-specific primers LS1 and LS2, respectively, and wherein F-AS indicates an allele-specific primer AS1 specific for HSV-1.

The non-complementary 5′ tail of the AS primers is underlined in the allele-specific primer, and the nucleotide specific for HSV-1 are shown in bold font.

Amplifications are performed in 96-well PCR plates in a 15 μl final reaction volume consisting of 20 ng DNA, 1×PCR buffer (Invitrogen), 100 ng/μl bovine serum albumin Fraction V (Sigma-Aldrich), 1.5 mM MgCl₂, 0.2 mM of each dNTP, 0.1 μM each locus-specific primer, 0.5 μM allele-specific primer and 0.375 U Tag DNA polymerase (Invitrogen).

Thermal amplification is performed in a 225-PTC thermal cycler (MJ Research, Bio-Rad) using the following conditions: 94° C. for 3 mins, followed by 15 cycles of 92° C. for 30 s, 58° C. for 30 s and 72° C. for 60 s, 5 cycles consisting of 92° C. for 10 s and 45° C. for 30 s and 15 cycles consisting of 92° C. for 10 s, 53° C. for 30 s and 72° C. for 5 s with a final extension step of 72° C. for 10 mins.

The presence of HSV-1 viral strain MP-S produces a 119-bp PCR product resulting from the F-AS and R-LS primers, while the presence of HSV-1 viral strain gC-39-R6 produces a 224-bp PCR product resulting from the F-LS and R-LS primers. Amplification of both the 119 and 224-bp PCR products indicates the presence of both HSV-1 viral strains. The absence of PCR product indicates the absence of both HSV-1 viral strains.

### EXAMPLE 17

### TSP Amplification to Identify Staphylococcus aureus

This example demonstrates the application of the method of the present invention to detect S. aureus in a sample.

S. aureus is a common cause of infections and a major public health threat causing a range of illnesses from minor skin infections to life threatening diseases such as pneumonia, toxic shock syndrome, acute respiratory distress syndrome (ARDS) and septicemia. Methicillin-resistant S. aureus (MRSA) strains, which are commonly multidrug resistant, present both a treatment and infection control challenge in hospital settings. Diagnostic tests to distinguish S. aureus infection are therefore important.

PCR primers for the TSP assay are based on the nucleotide sequence of the 16S rRNA gene of S. aureus (Genbank Accession No. AP009351).

Primers are as follows:

(SEQ ID NO: 105)

F-LS.SA: TGGAGCATGTGGTTTAATTCGA;

(SEQ ID NO: 106)

R-LS.SA: TGCGGGACTTAACCCAACA;

(SEQ ID NO: 107)

F-AS.SA: CGCTTACCAAATCTTGACAT,

wherein F-LS and R-LS are locus-specific primers LS1 and LS2, respectively, and wherein F-AS indicates an allele-specific primer AS1 specific for HSV-1.

The non-complementary 5′ tail of the AS primers is underlined in the allele-specific primer.

Amplifications are performed in 96-well PCR plates in a 15 μl final reaction volume consisting of 20 ng DNA, 1×PCR buffer (Invitrogen), 100 ng/μl bovine serum albumin Fraction V (Sigma-Aldrich), 1.5 mM MgCl₂, 0.2 mM of each dNTP, 0.1 μM each locus-specific primer, 0.5 μM allele-specific primer and 0.375 U Taq DNA polymerase (Invitrogen).

Thermal amplification is performed in a 225-PTC thermal cycler (MJ Research, Bio-Rad) using the following conditions: 94° C. for 3 mins, followed by 15 cycles of 92° C. for 30 s, 58° C. for 30 s and 72° C. for 60 s, 5 cycles consisting of 92° C. for 10 s and 45° C. for 30 s and 15 cycles consisting of 92° C. for 10 s, 53° C. for 30 s and 72° C. for 5 s with a final extension step of 72° C. for 10 mins.

The primers used in the first phase of amplification are directed to sequences conserved in all bacteria, whereas the second phase allele-specific primer is specific to S. aureus. As the 16S ribosomal gene is present in all bacteria, the presence of a 161-bp PCR product resulting from the F-LS and R-LS primers serves as a positive PCR internal control. Absence of this PCR product indicates a failed PCR assay, or the absence of nucleic acid from bacteria in the sample assayed. The presence of bacterium from the Staphylococcus genus is detected by the presence of a 124-bp PCR product resulting from the F-AS and R-LS primers, as well as the presence of the 161-bp PCR product.

We claim:

1. A method for detecting a polymorphism or mutation in nucleic acid, said method comprising:
(i) performing a polymerase chain reaction (PCR) under conditions sufficient to amplify a nucleic acid template comprising a polymorphism or mutation with one or more set(s) of first primers thereby producing a first amplification product, said set(s) of first primers capable of annealing selectively to a nucleic acid template comprising a polymorphism or mutation at a first temperature;
(ii) performing PCR under conditions sufficient to amplify the first amplification product with one or more second primer(s) or set(s) of second primers and/or with one or more of the primers from the set of first primers thereby producing a second amplification product comprising a sequence complementary to the allele-specific region and the tag region, said second primer(s) comprising an allele-specific region capable to annealing to the nucleic acid template and/or the first amplification product and a tag-region that does not anneal to the nucleic acid template, wherein said allele-specific region has a melting temperature (Tm) lower than the first primer and is not capable of annealing selectively to the template nucleic acid or the first amplification product at the first temperature and wherein the second primer is capable of annealing selectively to a nucleic acid comprising a sequence complementary to the allele-specific region and the tag region at about the first temperature, wherein said conditions comprise an annealing temperature suitable for annealing of the allele-specific region of the second primer(s) or set(s) of second primers to the first amplification product and/or the template nucleic acid and for the annealing of the first set of primers to the first amplification product and/or the template nucleic acid;
(iii) performing PCR under conditions sufficient to amplify the second amplification product to produce one or more third amplification product(s), said conditions comprising an annealing temperature suitable for annealing of the second primer(s) or set(s) of second primers to the second amplification product and for annealing of one or more primers from the set of first primers to the second amplification product but not for annealing of the allele specific region of the second primer(s) or set(s) of second primers to anneal selectively to the first amplification product at a detectable level, wherein the third amplification product(s) is/are amplified with the set(s) of second primers and/or a second primer and a first primer; and
(iv) detecting the third amplification product(s) with a detection means,
wherein detection of said third amplification product(s) is/are indicative of the polymorphism or mutation.

2. The method according to claim 1 wherein (i), (ii) and (iii) are performed in a single reaction vessel, and reagents suitable for performing PCR are provided in said reaction vessel, said reagents comprising the first primer or set of first primers and said second primer or set of second primers.

3. The method according to claim 1 wherein the conditions at (i) comprise an annealing temperature suitable for the set(s) of first primers to anneal selectively to the nucleic acid template but not for the allele-specific region of said second primer(s) or said set(s) of second primers to anneal selectively at a detectable level.

4. The method according to claim 1 wherein the second primer(s) comprise one or more 3′ terminal nucleotide(s) of the allele-specific region complementary to an allele of said polymorphism or mutation, wherein said primer(s) detectably produce the second amplification product and third amplification product only when said 3′ nucleotides anneal to the allele of said polymorphism or mutation.

5. The method according to claim 1 wherein the third amplification product is produced by PCR with a first primer and a second primer.

6. The method according to claim 1 additionally comprising detecting the first amplification product.

7. The method according to claim 1, wherein detection of the third amplification product produced by PCR with a first primer and a second primer homozygous for an allele of the polymorphism or mutation.

8. The method according to claim 6 wherein detection of the third amplification product produced by PCR with a first primer and a second primer and detection of the first amplification product is indicative of a nucleic acid heterozygous for an allele of the polymorphism or mutation.

9. The method according to claim 1 comprising performing a PCR at (ii) with a set of second primers, said set of second primers comprising (i) a second primer comprising one or more 3′ terminal nucleotide(s) of the allele-specific region complementary to an allele of said polymorphism or mutation, wherein said primer only detectably produces the second amplification product and the third amplification product when said 3′ nucleotides anneal to the allele of said polymorphism or mutation; and (ii) a second primer that anneals to nucleic acid adjacent to the polymorphism or mutation.

10. The method according to claim 9, wherein the 3′ terminal nucleotide(s) of the second primer at (i) anneal(s) to the allele and the third amplification product is produced by a PCR with the set of second primers, thereby indicating an allele of the polymorphism or mutation.

11. The method according to claim 9, wherein the 3′ terminal nucleotide(s) of the second primer at (i) do(es) not anneal(s) to the allele and the third amplification product is produced by PCR with the second primer at (ii) and a first primer, thereby indicating an allele of the polymorphism or mutation.

12. The method according to claim 1 comprising performing a PCR at (ii) with a plurality of second primers, wherein individual primers in said plurality comprise one or more 3′ nucleotide(s) complementary to a different allele of the polymorphism or mutation wherein said primers only detectably produce a second amplification product and third amplification product when said 3′ nucleotides anneal to the allele of said polymorphism or mutation, and wherein primers having different 3′ complementary nucleotide(s) also comprise a tag region having different molecular weights.

13. The method according to claim 12 comprising detecting the molecular weight of the third amplification product, wherein said molecular weight is indicative of an allele of the polymorphism or mutation.

14. The method according to claim 1 wherein the detection means comprises performing electrophoresis.

15. The method according to claim 14 wherein the electrophoresis is polyacrylamide gel electrophoresis or capillary electrophoresis.

16. The method according to claim 1 wherein the detection means detects the melting temperature of the third amplification product.

17. The method according to claim 1 comprising performing a PCR at (ii) with a plurality of second primers, wherein individual primers in said plurality comprise one or more 3′ nucleotide(s) complementary to a different allele of the polymorphism or mutation wherein said primers only detectably produce the second amplification product and the third amplification product when said 3′ nucleotides anneal to the allele of said polymorphism or mutation, and wherein primers comprising different 3′ nucleotide(s) also comprise a different detectable marker.

18. The method according to claim 17 comprising detecting the detectable marker, wherein detection of the detectable marker is indicative of the third amplification product.

19. The method according to claim 17 wherein the detectable marker is a fluorescent marker.

20. The method according to claim 1 comprising performing a PCR at (ii) with one or more second primer(s) or set(s) of second primers, said second primer(s) comprising an allele-specific region capable to annealing to nucleic acid adjacent to the polymorphism or mutation, and detecting the third amplification product comprises determining the melting temperature of the third amplification product, wherein the melting temperature of the third amplification product is indicative of the polymorphism or mutation.

21. The method according to claim 1 wherein the Tm of the allele-specific region of the second primer is at least about 10° C. less than the Tm of the first primer and/or the second primer.

22. The method according to claim 1 wherein the Tm of the first primer and Tm of the second primer is between about 60° C. and about 75° C.

23. The method according to claim 1 wherein the Tm of the allele specific region of the second primer is between about 35° C. and about 50° C.

24. The method according to claim 1 additionally comprising providing the nucleic acid.

25. The method according to claim 24 comprising providing the nucleic acid in a biological sample.

26. The method according to claim 1 additionally comprising providing a first set of primers and/or providing a second primer(s) or set(s) of second primers.

27. The method according to claim 1 wherein a first set of first primers is capable of annealing selectively to a genome of a polyploid organism to thereby detect a polymorphism or mutation in that genome.

28. A process for characterizing or identifying one or more individuals, isolates of an organism, cultivars of an organism, species or genera said process comprising performing the method according to claim 1 to detect one or more polymorphisms or mutations, wherein the one or more polymorphisms or mutations is(are) characteristic of the one or more individuals, isolates of an organism, cultivars of an organism, species or genera.

29. A process for identifying an infectious agent in a sample and/or for discriminating between infectious agents in a sample, said process comprising performing the method according to claim 1 to thereby detect one or more nucleic acid sequences of one or more infectious agents, wherein detection of said one or more nucleic acid sequences in the sample indicates the presence of an infectious agent in the sample and/or discriminates between infectious agents in the sample.

30. The process of claim 29, wherein the infectious agent is a virus, bacterium, fungus, protist, protozoan or parasite.

31. A process for identifying a subject having a trait or a disease or having a predisposition to developing a trait or disease, said process comprising performing the method according to claim 1, wherein the polymorphism or mutation is associated with said trait or disease and detection of said third amplification product is indicative of a subject having a trait or a disease or having a predisposition to developing a trait or disease.

32. The process of claim 31, wherein the polymorphism or mutation in is a methylenetetrahydrofolate reductase (MTHFR) gene of humans.

33. The process according to claim 31, wherein the polymorphism or mutation is in a plant gene associated with resistance of a plant to drought, frost, disease or a pest, or a plant gene associated with pre-harvest sprouting or nutritional quality of grain.

34. The process of claim 31 additionally comprising selecting a subject having the trait or a predisposition to developing the trait.

35. The process of claim 34 additionally comprising breeding a non-human subject having the trait or a predisposition to developing the trait.

36. A kit comprising:
(i) one or more set(s) of first primers, said set(s) of first primers capable of annealing selectively to a nucleic acid template comprising a polymorphism or mutation at a first temperature;
(ii) one or more second primer(s) or set(s) of second primers, said second primer(s) comprising an allele-specific region capable to hybridizing to the nucleic acid template and a tag-region that does not anneal to the nucleic acid template, wherein said allele-specific region has a melting temperature (Tm) lower than the first primer and is not capable of annealing selectively to the nucleic acid template at the first temperature and wherein the second primer is capable of annealing selectively to a nucleic acid comprising a sequence complementary to the allele-specific region and the tag region at about the first temperature; and
(iii) optionally, instructions for performing the method according to claim 1.

37. The kit according to claim 36 wherein the set(s) of second primers and the second primer(s) or set(s) of second primers are provided in a reaction vessel suitable for performing polymerase chain reaction (PCR).

38. A method of producing a set of primers, said method comprising:
(i) producing one or more set(s) of first primers, said set(s) of first primers capable of annealing selectively to a nucleic acid template comprising a polymorphism or mutation at a first temperature; and
(ii) producing one or more second primer(s) or set(s) of second primers, said second primer(s) comprising an allele-specific region capable to hybridizing to the nucleic acid template and a tag-region that does not anneal to the nucleic acid template, wherein said allele-specific region has a melting temperature (Tm) lower than the first primer and is not capable of annealing selectively to the nucleic acid template at the first temperature and wherein the second primer is capable of annealing selectively to a nucleic acid comprising a sequence complementary to the allele-specific region and the tag region at about the first temperature.

39. The method of claim 38 further comprises analyzing nucleotide sequence data to thereby determine a panel of candidate primers for inclusion in a set of primers.

40. The method of claim 38 further comprising determining a panel of first primer(s) and/or second primer(s) that provide discrimination between alleles in nucleic acid comprising a sequence homologous to the nucleic acid template.

41. The method of claim 38 further comprising selecting a panel of first primer(s) and/or second primer(s) that provide discrimination between alleles in nucleic acid comprising a sequence homologous to the nucleic acid template.

42. The method of claim 38 further comprising providing a panel of first primer(s) and/or second primer(s) that provide discrimination between alleles in nucleic acid comprising a sequence homologous to the nucleic acid template.

43. The method of claim 38 further comprising providing information pertaining to the sequences of a panel of first primer(s) and/or second primer(s) that provide discrimination between alleles in nucleic acid comprising a sequence homologous to the nucleic acid template.

44. A computer-readable medium comprising information pertaining to the sequences of a panel of first primer(s) and/or second primer(s) that provide discrimination between alleles in nucleic acid comprising a sequence homologous to the nucleic acid template, wherein said information is obtained by the method of claim 43.
